(19) United States
(12) Patent Application Publication
(10) Pub. No.: US 2009/0105263 A1
(43) Pub. Date: Apr. 23, 2009

(54) HETEROBICYCLIC COMPOUNDS AS GLUCOKINASE ACTIVATORS

(76) Inventors: Peter William Rodney Caulkett, Cheshire (GB); Darren McKerrecher, Cheshire (GB); Nicholas John Newcombe, Cheshire (GB); Kurt Gordon Pike, Cheshire (GB); Graeme Richard Robb, Cheshire (GB); Michael James Waring, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 12/066,967

(22) PCT Filed: Sep. 12, 2006
(86) PCT No.: PCT/GB2006/003382

§ 371 Date: Jun. 30, 2008

Related U.S. Application Data
(60) Provisional application No. 60/717,601, filed on Sep. 16, 2005.

(51) Int. Cl. A61K 31/4985; C07D 471/04; A61K 31/437; C07D 487/04; A61K 31/519

(52) U.S. Cl. 514/249; 546/113; 514/300; 544/280; 514/265.1; 544/350

(57) ABSTRACT

Compounds of Formula (I):

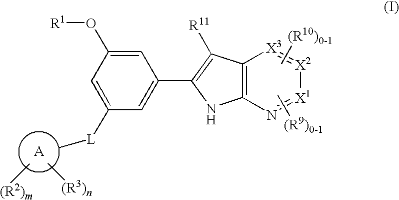

wherein R¹ to R¹¹, A and X¹ to X³ are as described in the specification, and their salts, are activators of glucokinase (GLK) and are thereby useful in the treatment of, for example, type 2 diabetes. Processes for preparing compounds of Formula (I) are also described.

The present invention relates to a group of fused imidazo-containing bicyclic compounds which are useful in the treatment or prevention of a disease or medical condition mediated through glucokinase (GLK or GK), leading to a decreased glucose threshold for insulin secretion. In addition the compounds are predicted to lower blood glucose by increasing hepatic glucose uptake. Such compounds may have utility in the treatment of Type 2 diabetes and obesity. The invention also relates to pharmaceutical compositions comprising said compounds and to methods of treatment of diseases mediated by GLK using said compounds.

In the pancreatic β-cell and liver parenchymal cells the main plasma membrane glucose transporter is GLUT2. Under physiological glucose concentrations the rate at which GLUT2 transports glucose across the membrane is not rate limiting to the overall rate of glucose uptake in these cells. The rate of glucose uptake is limited by the rate of phosphorylation of glucose to glucose-6-phosphate (G-6-P) which is catalysed by glucokinase (GLK) [1]. GLK has a high (6-10 mM) Km for glucose and is not inhibited by physiological concentrations of G-6-P [1]. GLK expression is limited to a few tissues and cell types, most notably pancreatic β-cells and liver cells (hepatocytes) [1]. In these cells GLK activity is rate limiting for glucose utilisation and therefore regulates the extent of glucose induced insulin secretion and hepatic glycogen synthesis. These processes are critical in the maintenance of whole body glucose homeostasis and both are dysfunctional in diabetes [2].

In one sub-type of diabetes, Maturity-Onset Diabetes of the Young Type 2 (MODY-2), the diabetes is caused by GLK loss of function mutations [3,4]. Hyperglycaemia in MODY-2 patients results from defective glucose utilisation in both the pancreas and liver [5]. Defective glucose utilisation in the pancreas of MODY-2 patients results in a raised threshold for glucose stimulated insulin secretion. Conversely, rare activating mutations of GLK reduce this threshold resulting in familial hyperinsulinism [6, 6a, 7]. In addition to the reduced GLK activity observed in MODY-2 diabetics, hepatic glucokinase activity is also decreased in type 2 diabetics [8]. Importantly, global or liver selective overexpression of GLK prevents or reverses the development of the diabetic phenotype in both dietary and genetic models of the disease [9-12]. Moreover, acute treatment of type 2 diabetics with fructose improves glucose tolerance through stimulation of hepatic glucose utilisation [13]. This effect is believed to be mediated through a fructose induced increase in cytosolic GLK activity in the hepatocyte by the mechanism described below [13].

Hepatic GLK activity is inhibited through association with GLK regulatory protein (GLKRP). The GLK/GLKRP complex is stabilised by fructose-6-phosphate (F6P) binding to the GLKRP and destabilised by displacement of this sugar phosphate by fructose-1-phosphate (F1P). F1P is generated by fructokinase mediated phosphorylation of dietary fructose. Consequently, GLK/GLKRP complex integrity and hepatic GLK activity is regulated in a nutritionally dependent manner as F6P is dominant in the post-absorptive state whereas F1P predominates in the post-prandial state. In contrast to the hepatocyte, the pancreatic β-cell expresses GLK in the absence of GLKRP. Therefore, β-cell GLK activity is regulated extensively by the availability of its substrate, glucose. Small molecules may activate GLK either directly or through destabilising the GLK/GLKRP complex. The former class of compounds are predicted to stimulate glucose utilisation in both the liver and the pancreas whereas the latter are predicted to act selectively in the liver. However, compounds with either profile are predicted to be of therapeutic benefit in treating Type 2 diabetes as this disease is characterised by defective glucose utilisation in both tissues.

GLK, GLKRP and the K_{ATP} channel are expressed in neurones of the hypothalamus, a region of the brain that is important in the regulation of energy balance and the control of food intake [14-18]. These neurones have been shown to express orectic and anorectic neuropeptides [15, 19, 20] and have been assumed to be the glucose-sensing neurones within the hypothalamus that are either inhibited or excited by changes in ambient glucose concentrations [17, 19, 21, 22]. The ability of these neurones to sense changes in glucose levels is defective in a variety of genetic and experimentally induced models of obesity [23-28]. Intracerebroventricular (icv) infusion of glucose analogues, that are competitive inhibitors of glucokinase, stimulate food intake in lean rats [29,30]. In contrast, icv infusion of glucose suppresses feeding [31]. Thus, small molecule activators of GLK may decrease food intake and weight gain through central effects on GLK. Therefore, GLK activators may be of therapeutic use in treating eating disorders, including obesity, in addition to diabetes. The hypothalamic effects will be additive or synergistic to the effects of the same compounds acting in the liver and/or pancreas in normalising glucose homeostasis, for the treatment of Type 2 diabetes. Thus the GLK/GLKRP system can be described as a potential “Diabesity” target (of benefit in both Diabetes and Obesity).

GLK is also expressed in specific entero-endocrine cells where it is believed to control the glucose sensitive secretion of the incretin peptides GIP (glucose-dependent insulinotropic polypeptide) and GLP-1 (Glucagon-Like Peptide-1) from gut K-cells and L-cells respectively (32, 33, 34). Therefore, small molecule activators of GLK may have additional beneficial effects on insulin secretion, b-cell function and survival and body weight as a consequence of stimulating GIP and GLP-1 secretion from these entero-endocrine cells.

In WO00/58293 and WO01/44216 (Roche), a series of benzylcarbamoyl compounds are described as glucokinase activators. The mechanism by which such compounds activate GLK is assessed by measuring the direct effect of such compounds in an assay in which GLK activity is linked to NADH production, which in turn is measured optically—see details of the in vitro assay described hereinafter. Compounds of the present invention may activate GLK directly or may activate GLK by inhibiting the interaction of GLKRP with GLK.

Further GLK activators have been described in WO03/095438 (substituted phenylacetamides, Roche), WO03/055482 (carboxamide and sulphonamide derivatives, Novo Nordisk), WO2004/002481 (arylcarbonyl derivatives, Novo Nordisk), and in WO03/080585 (amino-substituted benzoylaminoheterocycles, Banyu).

Our International application WO03/000267 describes a group of benzoyl amino pyridyl carboxylic acids which are activators of the enzyme glucokinase (GLK).

Our International application WO03/015774 describes compounds of the Formula (A):

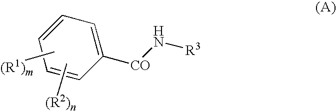

wherein R³ is a substituted heterocycle other than a carboxylic acid substituted pyridyl. One example was included having R³ as a bicyclic heterocycle (benzothiazolyl).

The amide functionality is a common feature of all of the above mentioned compounds.

International application WO 2004/016611 describes the use of imidazopyridine compounds as Inducible T cell kinase inhibitors. Such compounds were known for other uses (see inter alia EP 209707, U.S. Pat. No. 3,985,891 and WO 01/96336), but not as activators of glucokinase. International application WO 2005/63738 (Banyu) describes 2-heteroaryl substituted fused imidazole derivatives (such as 2-heteroaryl substituted benzimidazole compounds) which are glucokinase activators. Our co-pending application PCT/GB2006/001842 describes fused imidazole-containing bicyclic compounds.

We have surprisingly found that fused pyrrole-containing bicyclic compounds, such as pyrrolopyridine and pyrrolopyrazine, not containing central amide functionality are GLK activators. The compounds of the invention have generally good potency for the GLK enzyme, and may have advantageous toxicological and/or physical properties (including, for example, higher aqueous solubility, higher permeability, and/or lower plasma protein binding) which may make them particularly suitable for use in the treatment or prevention of a disease or medical condition mediated through GLK.

Thus, according to the first aspect of the invention there is provided a compound of Formula (I):

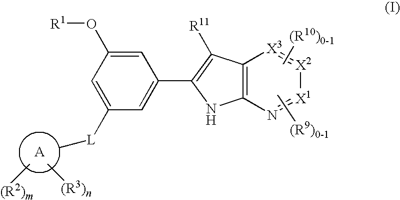

wherein:
Ring A is selected from phenyl and HET-1;
X¹, X² and X³ are each independently CH or N, with the proviso that only one of X¹, X² and X³ may be N;
L is a linker selected from —O— and -(1-3C)alkylO— (wherein the oxygen is directly attached to the central phenyl ring which is substituted by —OR¹);
R¹ is selected from (1-6C)alkyl, (2-6C)alkenyl, (2-6C)alkynyl, (3-6C)cycloalkyl, (3-6C)cycloalkyl(1-6C)alkyl, aryl(1-6C)alkyl, HET-1a and HET-1a-(1-6C)alkyl;
wherein any alkyl, alkenyl, alkynyl, cycloalkyl, aryl or HET-1a group in any definition of;
R¹ may optionally be substituted on an available carbon atom 1 or more halo and/or with a substituent selected from hydroxy, (1-4C)alkoxy, (1-6C)alkylamino, di(1-6C)alkylamino, (C_{q}H_{2q+2-a}Fₐ)—O— (wherein q=1 to 4 and a=1 to 3), (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)alkylaminocarbonyl, di(1-6C)alkylaminocarbonyl, carboxy and cyano; and/or may be substituted on an available nitrogen atom (provided the nitrogen is not thereby quaternised) by a substituent selected from (1-6C)alkylsulfonyl, (1-6C)alkylaminosulfonyl, di(1-6C)alkylaminosulfonyl, (1-6C)alkylaminocarbonyl and di(1-6C)alkylaminocarbonyl;
HET-1 and HET-1a are independently a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group;
R² is selected from —C(O)NR⁴R⁵, —SO₂NR⁴R⁵, —S(O)ₚR⁴ and HET-2;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available nitrogen atom (provided the nitrogen is not thereby quaternised) by a substituent selected from R⁶ and/or on an available carbon atom by 1 or 2 substituents independently selected from R⁷;
R³ is selected from halo, fluoromethyl, difluoromethyl, trifluoromethyl, methyl, (1-4C)alkoxy, carboxy and cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷), cyano, —NR^{4′}R^{5′} and —C(O)NR⁵R⁵], fluoromethyl, difluoromethyl, trifluoromethyl, (3-6C)cycloalkyl (optionally substituted with 1 group selected from R⁷), (2-4C)alkenyl (optionally substituted with 1 group selected from R⁷), (2-4C)alkynyl (optionally substituted with 1 group selected from R⁷), and HET-2;
R⁵ is (independently at each occurrence) selected from hydrogen, (1-4C)alkyl and (3-6C)cycloalkyl;
or R⁴ and R⁵ together with the nitrogen atom to which they are attached may form a heterocyclyl ring system as defined by HET-3;
R^{4′} and R^{5′} are independently selected from hydrogen and (1-4C)alkyl; or
R^{4′} and R^{5′} together with the nitrogen atom to which they are attached may form a 4- to 6-membered saturated ring;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4 to 7 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸;
when R⁸ is a substituent on carbon it is selected from halo, —OR⁵, (1-4C)alkyl, (2-4C)alkenyl, (2-4C)alkynyl, trifluoromethyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
when R⁸ is a substituent on nitrogen it is selected from (1-4C)alkyl, —C(O)(1-4C)alkyl, —C(O)NR⁴R⁵, HET-3 (wherein said ring is unsubstituted), (1-4C)alkoxy(2-4C)alkyl, hydroxy(2-4C)alkyl and —S(O)pR⁵;
R⁹ is selected from (1-4C)alkyl, halo, cyano, hydroxy(1-4C)alkyl, dihydroxy(2-4C)alkyl, (1-4C)alkoxy(1-4C)alkyl, di(1-4C)alkoxy(2-4C)alkyl, (1-4C)alkylS(O)ₚ(1-4C)alkyl, amino(1-4C)alkyl, (1-4C)alkylamino(1-4C)alkyl, di(1-4C)alkylamino(1-4C)alkyl, (1-4C)alkylcarbonylamino, (1-4C)alkylcarbonyl-N-[(1-4C)alkyl]amino, (1-4C)alkylaminocarbonyl and di(1-4C)alkylaminocarbonyl;
R¹⁰ is selected from methoxy, methyl and halo;
R¹¹ is selected from hydrogen and (1-4C)alkyl;
p is (independently at each occurrence) 0, 1 or 2;
m is 0 or 1;
n is 0, 1 or 2;
or a salt thereof.

According to another aspect of the invention, there is provided a compound of formula (I) as hereinbefore defined, wherein:

Ring A is selected from phenyl and HET-1;
X¹, X² and X³ are each independently CH or N, with the proviso that only one of X¹, X² and X³ may be N;
L is a linker selected from —O— and -(1-3C)alkylO— (wherein the oxygen is directly attached to the central phenyl ring);
R¹ is selected from (1-6C)alkyl, (2-6C)alkenyl, (2-6C)alkynyl, (3-6C)cycloalkyl, (3-6C)cycloalkyl(1-6C)alkyl, aryl(1-6C)alkyl, HET-1a and HET-1a-(1-6C)alkyl;
wherein any alkyl, alkenyl, alkynyl, cycloalkyl, aryl or HET-1a group in any definition of R¹ may optionally be substituted on an available carbon atom 1 or more halo and/or with a substituent selected from hydroxy, (1-4C)alkoxy, (1-6C)alkylamino, di(1-6C)alkylamino, (C_{q}H_{2q+2-a}Fₐ)—O— (wherein q=1 to 4 and a=1 to 3), (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)alkylaminocarbonyl, di(1-6C)alkylaminocarbonyl, carboxy and cyano; and/or may be substituted on an available nitrogen atom (provided the nitrogen is not thereby quaternised) by a substituent selected from (1-6C)alkylsulfonyl, (1-6C)alkylaminosulfonyl, di(1-6C)alkylaminosulfonyl, (1-6C)alkylaminocarbonyl and di(1-6C)alkylaminocarbonyl;
HET-1 and HET-1a are independently a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group;
R² is selected from —C(O)NR⁴R⁵, —SO₂NR⁴R⁵, —S(O)ₚR⁴ and HET-2;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available nitrogen atom (provided the nitrogen is not thereby quaternised) by a substituent selected from R⁶ and/or on an available carbon atom by 1 or 2 substituents independently selected from R⁷;
R³ is selected from halo, fluoromethyl, difluoromethyl, trifluoromethyl, methyl, (1-4C)alkoxy, carboxy and cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁵ is, independently at each occurrence, hydrogen or (1-4C)alkyl;
or R⁴ and R⁵ together with the nitrogen atom to which they are attached may form a heterocyclyl ring system as defined by HET-3;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4 to 7 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸;
when R⁸ is a substituent on carbon it is selected from —OR⁵, (1-4C)alkyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
when R⁸ is a substituent on nitrogen it is selected from (1-4C)alkyl, —C(O)(1-4C)alkyl, —C(O)NR⁴R⁵, HET-3 (wherein said ring is unsubstituted), (1-4C)alkoxy(2-4C)alkyl, hydroxy(2-4C)alkyl and —S(O)pR⁵;
R⁹ is selected from (1-4C)alkyl, halo, hydroxy(1-4C)alkyl, dihydroxy(2-4C)alkyl, (1-4C)alkoxy(1-4C)alkyl, di(1-4C)alkoxy(2-4C)alkyl, (1-4C)alkylS(O)ₚ(1-4C)alkyl, amino(1-4C)alkyl, (1-4C)alkylamino(1-4C)alkyl, di(1-4C)alkylamino(1-4C)alkyl, (1-4C)alkylcarbonylamino, (1-4C)alkylcarbonyl-N-[(1-4C)alkyl]amino, (1-4C)alkylaminocarbonyl, and di(1-4C)alkylaminocarbonyl;
R¹⁰ is selected from methoxy, methyl and halo;
R¹¹ is selected from hydrogen and (1-4C)alkyl;
p is (independently at each occurrence) 0, 1 or 2;
m is 0 or 1;
n is 0, 1 or 2;
or a salt thereof.

It will be understood that where L is -(1-3C)alkylO—, the alkyl chain may be linear or branched; this definition of L thus encompasses, for example —CH₂—CH₂—O— and —CH₂—CH(Me)-O—.

It will be understood that a (1-6C)alkyl, alkenyl or alkynyl chain in any definition of R¹ may be linear or branched.

It will be understood that when R⁴ is —C(O)NR⁵R⁵, each R⁵ is independently selected from hydrogen and (1-4C)alkyl, and therefore this definition of R⁴ includes (but is not limited to) —CONH₂, —CONHMe, —CONMe₂ and —CONMeEt.

It will be understood that where a compound of the formula (I) contains more than one HET-2 ring, they may be the same or different.

It will be understood that where a compound of the formula (I) contains more than one HET-3 ring, they may be the same or different.

It will be understood that where a compound of the formula (I) contains more than one group R⁴, they may be the same or different.

It will be understood that where a compound of the formula (I) contains more than one group R⁵, they may be the same or different.

It will be understood that where a compound of the formula (I) contains more than one group R⁷, they may be the same or different.

It will be understood that where a compound of the formula (I) contains more than one group R⁸, they may be the same or different.

A similar convention applies for all other groups and substituents on a compound of formula (I) as hereinbefore defined.

It will be understood that R¹ may be substituted on more than one available carbon and/or nitrogen atom by the listed optional substituents, which may be the same or different.

It will be understood that HET-2 may be substituted on more than one available carbon and/or nitrogen atom by the listed optional substituents, which may be the same or different.

It will be understood that HET-3 may be substituted on more than one available carbon and/or nitrogen atom by the listed optional substituents, which may be the same or different.

It will be understood that R⁹ and R¹⁰ may only be substituents on a ring carbon atom (ie where X=C).

It will be understood that when A is HET-1, substitution of Ring A by R² and/or R³ is not intended to lead to unstable structures.

In general, it will be understood that substitution on any particular group is not intended to include unstable structures, for example those wherein two heteroatoms (such as O, N and S) are attached to the same carbon atom. Substitution on a nitrogen atom will be understood not to lead to quaternisation of said nitrogen atom.

Compounds of Formula (I) may form salts which are within the ambit of the invention. Pharmaceutically acceptable salts are preferred although other salts may be useful in, for example, isolating or purifying compounds.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

In another aspect, the invention relates to compounds of formula (I) as hereinabove defined or to a pharmaceutically acceptable salt.

In another aspect, the invention relates to compounds of formula (I) as hereinabove defined or to a pro-drug thereof. Suitable examples of pro-drugs of compounds of formula (I) are in-vivo hydrolysable esters of compounds of formula (I). Therefore in another aspect, the invention relates to compounds of formula (I) as hereinabove defined or to an in-vivo hydrolysable ester thereof.

In this specification the generic term “alkyl” includes both straight-chain and branched-chain alkyl groups. However references to individual alkyl groups such as “propyl” are specific for the straight chain version only and references to individual branched-chain alkyl groups such as t-butyl are specific for the branched chain version only. For example, “(1-4C)alkyl” includes methyl, ethyl, propyl, isopropyl and t-butyl. An analogous convention applies to other generic terms.

It will be appreciated that, where definitions of heterocyclyl groups HET-1, HET-1a, HET-2 and/or HET-3 encompass heteroaryl rings which may be substituted on nitrogen, such substitution may not result in charged quaternary nitrogen atoms. It will be appreciated that the definitions of HET-1 to HET-3 are not intended to include any 0-0, O—S or S—S bonds. It will be appreciated that the definitions of HET-1 to HET-3 are not intended to include unstable structures.

If not stated elsewhere, suitable optional substituents for a particular group are those as stated for similar groups herein.

Examples of (1-4C)alkyl include methyl, ethyl, propyl, isopropyl, butyl and tert-butyl; examples of (1-6C)alkyl include (1-4C)alkyl, pentyl and hexyl; examples of (2-4C)alkenyl and (2-6C)alkenyl include vinyl, prop-2-enyl, prop-1-enyl, but-2-enyl and isobutenyl; examples of (2-4C)alkynyl and (2-6C)alkynyl include ethynyl, prop-1pynyl, prop-2-ynyl, and but-2-ynyl; examples of (3-6C)cycloalkyl include cyclopropyl, cyclobutyl, cyclopentyl and cyclohexyl; examples of (3-6C)cycloalkyl(1-6C)alkyl include cyclopropylmethyl, cyclobutylethyl, cyclopentylpropyl and cyclohexylmethyl; examples of halo include fluoro, chloro, bromo and iodo; examples of hydroxy(1-4C)alkyl include hydroxymethyl, 1-hydroxyethyl, 2-hydroxyethyl, 2-hydroxypropyl, 3-hydroxypropyl, 1-hydroxyisopropyl and 4-hydroxybutyl; examples of dihydroxy(2-4C)alkyl include 1,2-dihydroxyethyl, 1,2-dihydroxypropyl, 1,3-dihydroxypropyl, 2,3-dihydroxypropyl, 1,2-dihydroxybutyl, 1,3-dihydroxybutyl, 2,3-dihydroxybutyl, 3-4dihydroxybutyl and 2,4-dihydroxybutyl; examples of (1-4C)alkoxy(1-4C)alkyl include methoxymethyl, ethoxymethyl, tert-butoxymethyl, 2-methoxyethyl, 2-ethoxyethyl, methoxypropyl, 2-methoxypropyl and methoxybutyl; examples of di(1-4C)alkoxy(2-4C)alkyl include 1,2-dimethoxyethyl, 1-methoxy-2-ethoxy-ethyl, 1,2-dimethoxypropyl, 1,3-dimethoxypropyl, 2,3-dimethoxypropyl, 1,2-dimethoxybutyl, 2,3-dimethoxybutyl, 2,4-dimethoxybutyl and 3,4-dimethoxybutyl; examples of (1-4C)alkylS(O)p(1-4C)alkyl include methylsulfinylmethyl, ethylsulfinylmethyl, ethylsulfinylethyl, methylsulfinylpropyl, methylsulfinylbutyl, methylsulfonylmethyl, ethylsulfonylmethyl, ethylsulfonylethyl, methylsulfonylpropyl, methylsulfonylbutyl, methylthiomethyl, ethylthiomethyl, ethylthio ethyl, methylthiopropyl, and methylthiobutyl; examples of amino(1-4C)alkyl include aminomethyl, aminoethyl, 2-aminopropyl, 3-aminopropyl, 1-aminoisopropyl and 4-aminobutyl; examples of (1-4C)alkylamino(1-4C)alkyl include (N-methyl)aminomethyl, (N-ethyl)aminomethyl, 1-((N-methyl)amino)ethyl, 2-((N-methyl)amino)ethyl, (N-ethyl)aminoethyl, (N-methyl)aminopropyl, and 4-((N-methyl)amino)butyl; examples of di(1-4C)alkylamino(1-4C)alkyl include dimethylaminomethyl, methyl(ethyl)aminomethyl, methyl(ethyl)aminoethyl, (N,N-diethyl)aminoethyl, (N,N-dimethyl)aminopropyl and (N,N-dimethyl)aminobutyl; examples of (1-4C)alkylamino include methylamino, ethylamino, propylamino, isopropylamino, butylamino and tert-butylamino; examples of di(1-4C)alkylamino include dimethylamino, methyl(ethyl)amino, diethylamino, dipropylamino, di-isopropylamino and dibutylamino; examples of —C(O)(1-4C)alkyl include methylcarbonyl, ethylcarbonyl, propylcarbonyl and tert-butyl carbonyl; examples of (1-6C)alkylsulfonyl include methylsulfonyl, ethylsulfonyl, propylsulfonyl, isopropylsulfonyl and tert-butylsulfonyl; examples of (1-6C)alkylsulfonylamino include methylsulfonylamino, ethylsulfonylamino, propylsulfonylamino, isopropylsulfonylamino and tert-butylsulfonylamino; examples of (1-6C)alkylsulfonyl-N-[(1-6C)alkyl]amino include methylsulfonyl-N-(methyl)amino, ethylsulfonyl-N-(methyl)amino, propylsulfonyl-N-(methyl)amino, isopropylsulfonyl-N-(methyl)amino and tert-butylsulfonyl-N-(methyl)amino; examples of (1-6C)alkylaminosulfonyl include methylaminosulfonyl, ethylaminosulfonyl, propylaminosulfonyl, isopropylaminosulfonyl and tert-butylaminosulfonyl; examples of di(1-6C)alkylaminosulfonyl include dimethylaminosulfonyl, diethylaminosulfonyl, methyl(propyl)aminosulfonyl, diisopropylaminosulfonyl and tert-butyl(methyl)aminosulfonyl; examples of (1-6C)alkylaminocarbonyl include (1-4C)alkylaminocarbonyl such as methylaminocarbonyl, ethylaminocarbonyl, propylaminocarbonyl, isopropylaminocarbonyl and tert-butylaminocarbonyl; examples of di(1-6C)alkylaminocarbonyl include di(1-4C)alkylaminocarbonyl such as dimethylaminocarbonyl, diethylaminocarbonyl, methyl(propyl)aminocarbonyl, diisopropylaminocarbonyl and tert-butyl(methyl)aminocarbonyl; examples of (1-6C)alkylcarbonylamino include (1-4C)alkylcarbonylamino such as methylcarbonylamino, ethylcarbonylamino, propylcarbonylamino, isopropylcarbonylamino and tert-butylcarbonylamino; examples of (1-6C)alkylcarbonyl-N-[(1-6C)alkyl]amino include (1-4C)alkylcarbonyl-N-[(1-4C)alkyl]amino methylcarbonyl-N-(methyl)amino, ethylcarbonyl-N-(methyl)amino, propylcarbonyl-N-(methyl)amino, isopropylcarbonyl-N-(methyl)amino and tert-butylcarbonyl-N-(methyl)amino.

Aryl is phenyl or naphthyl, preferably phenyl.

Examples of aryl(1-6C)alkyl include benzyl, phenethyl, phenylpropyl and naphthylmethyl.

Suitable examples of HET-1 and HET-1a as a 5- or 6-membered, C-linked heteroaryl ring as hereinbefore defined, include thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl and triazolyl.

When A is HET-1, further suitable values include pyridyl, pyrazinyl, pyrimidinyl and pyridazinyl.

When A is HET-1, a further suitable value is pyrimidinyl.

Suitable examples of HET-1-(1-6C)alkyl and HET-1a-(1-6C)alkyl include any of the above values for HET-1 and HET-1a in combination with any of the above values for (1-6C)alkyl.

It will be understood that HET-2 can be a saturated, or partially or fully unsaturated ring.

Suitable examples of HET-2 include azetidinyl, furyl, thienyl, thiazolyl, isothiazolyl, thiadiazolyl, pyridyl, pyrazinyl, pyridazinyl, pyrazolyl, imidazolyl, pyrimidinyl, oxazolyl, isoxazolyl, oxadiazolyl, morpholino, morpholinyl, piperidinyl, piperazinyl, thiomorpholino, thiomorpholinyl, pyrrolyl, pyrrolidinyl, pyrrolidonyl, 2,5-dioxopyrrolidinyl, tetrahydrothienyl, 1-oxotetrahydrothienyl, 1,1-dioxotetrahydrothienyl, 2-oxoimidazolidinyl, 2,4-dioxoimidazolidinyl, 2-oxo-1,3,4-(4-triazolinyl), 2-oxazolidinonyl, 2-oxotetrahydrofuranyl, tetrahydrofuranyl, tetrahydropyranyl, 1,1-dioxothiomorpholino, 1,3-dioxolanyl, 1,2,4-triazolyl, 1,2,3-triazolyl, pyranyl, and 4-pyridonyl.

It will be understood that HET-2 may be linked by any appropriate available C or N atom, therefore for example, for HET-2 as “imidazolyl” includes 1-, 2-, 4- and 5-imidazolyl.

Suitable examples of HET-3 as a 4-6 membered saturated or partially unsaturated heterocyclic ring are morpholino, thiomorpholino (and versions thereof wherein the sulfur is oxidised to an SO or S(O)₂ group), piperidinyl, piperazinyl, pyrrolidinyl and azetidinyl. A suitable example of HET-3 as a 7-membered saturated or partially unsaturated heterocyclic ring is homopiperazinyl, homo-morpholino, homo-thiomorpholino (and versions thereof wherein the sulfur is oxidised to an SO or S(O)₂ group) and homo-piperidinyl.

It is to be understood that, insofar as certain of the compounds of Formula (I) defined above may exist in optically active or racemic forms by virtue of one or more asymmetric carbon atoms, the invention includes in its definition any such optically active or racemic form which possesses the property of stimulating GLK directly or inhibiting the GLK/GLKRP interaction. The synthesis of optically active forms may be carried out by standard techniques of organic chemistry well known in the art, for example by synthesis from optically active starting materials or by resolution of a racemic form. It is also to be understood that certain compounds may exist in tautomeric forms and that the invention also relates to any and all tautomeric forms of the compounds of the invention which activate GLK.

It is also to be understood that certain compounds of the formula (I) and salts thereof can exist in solvated as well as unsolvated forms such as, for example, hydrated forms. It is to be understood that the invention encompasses all such solvated forms which activate GLK.

In one aspect, there is provided a compound of formula (IA) or a salt thereof;

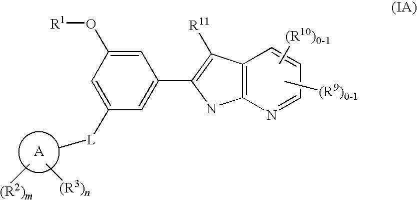

wherein R¹, R², R³, R⁹, R¹⁰, R¹¹, m, n, A and L are as defined for formula (I). It will be understood that the compound of formula (IA) is a compound of formula (I) wherein X¹, X² and X³ are all CH.

In another aspect, there is provided a compound of formula (IB) or a salt thereof;

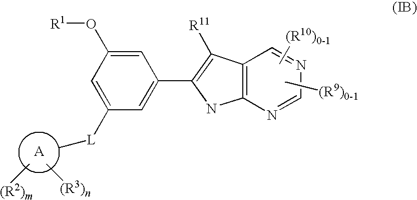

wherein R¹, R², R³, R⁹, R¹⁰, R¹¹, m, n, A and L are as defined for formula (I). It will be understood that the compound of formula (IB) is a compound of formula (I) wherein X¹ and X³ are both CH and X² is N.

In another aspect, there is provided a compound of formula (IC) or a salt thereof;

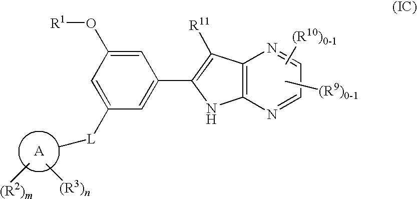

wherein R¹, R², R³, R⁹, R¹⁰, R¹¹, m, n, A and L are as defined for formula (I). It will be understood that the compound of formula (IB) is a compound of formula (I) wherein X¹ and X² are both CH and X³ is N.

It will be appreciated that any aspect or embodiment hereinbefore or after referring to a compound of formula (I) is intended to apply equally to a compound of formula (IA) or a compound of formula (IB) or a compound of formula (IC), even where not explicitly stated.

In one embodiment of the invention are provided compounds of formula (I), in an alternative embodiment are provided pharmaceutically-acceptable salts of compounds of formula (I), (IA), (IB) and (IC), in a further alternative embodiment are provided in-vivo hydrolysable esters of compounds of formula (I), (IA), (IB) and (IC), and in a further alternative embodiment are provided pharmaceutically-acceptable salts of in-vivo hydrolysable esters of compounds of formula (I), (IA), (IB) and (IC).

Preferred values of each variable group are as follows. Such values may be used where appropriate with any of the values, definitions, claims, aspects or embodiments defined hereinbefore or hereinafter. In particular, each may be used as an individual limitation on the broadest definition of formula (I), (IA), (IB) and/or (IC). Further, each of the following values may be used in combination with one or more of the other following values to limit the broadest definition of formula (I), (IA), (IB) and/or (IC).

(1) R¹ is optionally substituted (1-6C)alkyl, preferably optionally substituted branched (1-6C)alkyl
(2) R¹ is optionally substituted (2-6C)alkenyl
(3) R¹ is optionally substituted (2-6C)alkynyl
(4) R¹ is optionally substituted (3-6C)cycloalkyl
(5) R¹ is optionally substituted (3-6C)cycloalkyl(1-6C)alkyl
(6) R¹ is optionally substituted aryl(1-6C)alkyl
(7) R¹ is optionally substituted HET-1a
(8) R¹ is optionally substituted HET-1a-(1-6C)alkyl
(9) R¹ is optionally substituted by hydroxy
(10) R¹ is optionally substituted by (1-4C)alkoxy
(11) R¹ is optionally substituted by 1 or more halo, or by (CₙH₂ₙ₊₂₋ₐFₐ)—O— (wherein n=1 to 4 and a=1 to 3), particularly R¹ is (1-4C)alkyl optionally substituted by 1 or more halo, or by (CₙH₂ₙ₊₂₋ₐFₐ)—O—
(12) R¹ is optionally substituted by (1-6C)alkylamino or di(1-6C)alkylamino
(13) R¹ is optionally substituted by carboxy or cyano
(14) R¹ is optionally substituted on carbon by a substituent selected from (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)aminocarbonyl and di(1-6C)alkylaminocarbonyl
(15) R¹ is hydroxyisopropyl and the configuration is preferably (S), that is —O—R¹ is the group:

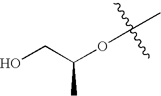

(16) R¹ is methoxyisopropyl and the configuration is preferably (S), that is —O—R¹ is the group:

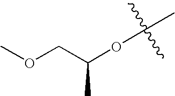

(17) R¹ is isopropyl
(18) Ring A is phenyl

### (19) Ring A is HET-1

(20) Ring A is HET-1 and HET-1 is a fully unsaturated (aromatic) heterocyclic ring
(21) Ring A is phenyl or HET-1 and HET-1 is a fully unsaturated (aromatic) heterocyclic ring
(22) Ring A is HET-1 and HET-1 is selected from pyridyl, pyrimidinyl, pyrazinyl and pyridazinyl
(23) Ring A is HET-1 and HET-1 is selected from pyridyl, pyrimidinyl and pyrazinyl
(24) Ring A is HET-1 and HET-1 is selected from pyridyl and pyrazinyl
(25) Ring A is selected from phenyl, pyridyl, pyrimidinyl and pyrazinyl
(26) Ring A is selected from phenyl, pyridyl and pyrazinyl
(27) Ring A is phenyl or pyrimidinyl

### (28) L is —O—

### (29) L is —O—CH₂—

### (30) L is —O—CH₂—CH₂—

### (31) L is —O—CH₂—CH₂—CH₂—

(32) HET-1 is a 4-membered heterocyclyl ring
(33) HET-1 is a 5- or 6-membered heterocyclyl ring
(34) HET-1 is a 5-membered heterocyclyl ring
(35) HET-1 is a 6-membered heterocyclyl ring

### (36) HET-1 is N-linked

### (37) HET-1 is C-linked

(38) HET-1a is a 4-membered heterocyclyl ring
(39) HET-1a is a 5- or 6-membered heterocyclyl ring
(40) HET-1a is a 5-membered heterocyclyl ring
(41) HET-1a is a 6-membered heterocyclyl ring

### (42) HET-1a is N-linked

### (43) HET-1a is C-linked

### (44) R² is —C(O)NR⁴R⁵

### (45) R² is —SO₂NR⁴R⁵

### (46) R² is —S(O)ₚR⁴

### (47) R² is HET-2

(48) R² is —C(O)NR⁴R⁵, R⁴ and R⁵ together with the nitrogen atom to which they are attached form a heterocyclyl ring system as defined by HET-3, selected from morpholino, thiomorpholino (and versions thereof wherein the sulfur is oxidised to an SO or S(O)₂ group), piperidinyl, piperazinyl, pyrrolidinyl and azetidinyl
(49) R² is —SO₂Me or —C(O)NR⁴R⁵ wherein —C(O)NR⁴R⁵ is a HET-3 ring, particularly an azetidinyl ring
(50) HET-2 is a 4-membered heterocyclyl ring
(51) HET-2 is a 5- or 6-membered heterocyclyl ring
(52) HET-2 is a 5-membered heterocyclyl ring
(53) HET-2 is a 6-membered heterocyclyl ring

### (54) HET-2 is N-linked

### (55) HET-2 is C-linked

(56) HET-2 is unsubstituted
(57) HET-2 is substituted on a carbon atom with 1 substituent selected from R⁷
(58) HET-2 is substituted on a nitrogen atom with 1 substituent selected from R⁶
(59) R³ is selected from halo, (1-4C)alkoxy (such as methoxy) and methyl
(60) R³ is selected from fluoromethyl, difluoromethyl and trifluoromethyl
(61) R³ is selected from carboxy and cyano
(62) R⁴ is hydrogen
(63) R⁴ is optionally substituted (1-4C)alkyl
(64) R⁴ is (1-4C)alkyl substituted by HET-2
(65) R⁴ is (1-4C)alkyl substituted with —OR⁵, particularly hydroxy or methoxy
(66) R⁴ is (1-4C)alkyl substituted with —SO₂R⁵
(67) R⁴ is (1-4C)alkyl substituted with (3-6C)cycloalkyl (optionally substituted with 1 group selected from R⁷)
(68) R⁴ is (1-4C)alkyl substituted with —C(O)NR⁵R⁵
(69) R⁴ is (3-6C)cycloalkyl (optionally substituted with 1 group selected from R⁷)

### (70) R⁴ is HET-2

(71) R⁵ is hydrogen

### (72) R⁵ is (1-4C)alkyl

(73) R⁴ and R⁵ together with the nitrogen atom to which they are attached form a heterocyclyl ring system as defined by HET-3
(74) R⁴ and R⁵ together with the nitrogen atom to which they are attached form a heterocyclyl ring system as defined by HET-3, selected from morpholino, thiomorpholino (and versions thereof wherein the sulfur is oxidised to an SO or S(O)₂ group), piperidinyl, piperazinyl, pyrrolidinyl, azetidinyl, homopiperazinyl, homo-morpholino, homo-thiomorpholino (and versions thereof wherein the sulfur is oxidised to an SO or S(O)₂ group) and homo-piperidinyl
(75) R⁴ and R⁵ together with the nitrogen atom to which they are attached form a heterocyclyl ring system as defined by HET-3, selected from morpholino, thiomorpholino (and versions thereof wherein the sulfur is oxidised to an SO or S(O)₂ group), piperidinyl, piperazinyl, pyrrolidinyl and azetidinyl
(76) R⁴ and R⁵ together with the nitrogen atom to which they are attached form a heterocyclyl ring system as defined by HET-3, selected from piperidinyl, piperazinyl, pyrrolidinyl and azetidinyl
(77) R⁴ is (1-4C)alkyl substituted with —NR^{4′}R^{5′}
(78) R⁴ is (1-4C)alkyl substituted with —NR^{4′}R^{5′} and R^{4′} and R^{5′} are each independently hydrogen or (1-4C)alkyl, particularly hydrogen or methyl
(79) R⁴ is (1-4C)alkyl substituted with cyano

### (80) R⁴ is (2-4C)alkenyl

### (81) R⁴ is (2-4C)alkynyl

(82) R⁶ is selected from (1-4C)alkyl, (1-4C)alkoxy(1-4C)alkyl and hydroxy(1-4C)alkyl
(83) R⁶ is selected from —C(O)(1-4C)alkyl, —C(O)NR⁴R⁵, and —S(O)pR⁵
(84) R⁷ is selected from (1-4C)alkyl, (1-4C)alkoxy(1-4C)alkyl and hydroxy(1-4C)alkyl
(85) R⁷ is selected from —OR⁵, —C(O)(1-4C)alkyl, —C(O)NR⁴R⁵, and —S(O)pR⁵
(86) R⁷ is selected from —OR⁵ (wherein R⁵ is hydrogen or (1-4C)alkyl) and hydroxy(1-4C)alkyl
(87) HET-3 is 4-membered ring
(88) HET-3 is a 5-membered ring
(89) HET-3 is a 6-membered ring
(90) HET-3 is a 7-membered ring
(91) HET-3 is unsubstituted
(92) HET-3 is substituted (preferably on a carbon atom) with 1 substituent R⁸
(93) R⁸ is a substituent on carbon and is selected from —OR⁵, (1-4C)alkyl, (1-4C)alkoxy(1-4C)alkyl and hydroxy(1-4C)alkyl
(94) R⁸ is a substituent on carbon and is selected from halo, —OR⁵, (1-4C)alkyl, (1-4C)alkoxy(1-4C)alkyl and hydroxy(1-4C)alkyl
(95) R⁸ is substituent on carbon and is selected from —C(O)(1-4C)alkyl, —C(O)NR⁴R⁵, (1-4C)alkylamino and di(1-4C)alkylamino
(96) R⁸ is substituent on nitrogen and is selected from (1-4C)alkyl, —C(O)(1-4C)alkyl, and —C(O)NR⁴R⁵
(97) R⁸ is substituent on nitrogen and is selected from HET-3 (wherein said ring is unsubstituted), (1-4C)alkoxy(2-4C)alkyl, hydroxy(2-4C)alkyl and —S(O)pR⁵
(98) R⁸ is selected from HET-3 (wherein said ring is unsubstituted)

### (99) R⁸ is —S(O)pR⁵

(100) R⁵ is selected from methoxy and methyl
(101) R⁹ is selected from (1-4C)alkyl and halo
(102) R⁹ is selected from hydroxy(1-4C)alkyl, dihydroxy(2-4C)alkyl, (1-4C)alkoxy(1-4C)alkyl and di(1-4C)alkoxy(2-4C)alkyl
(103) R⁹ is selected from (1-4C)alkylS(O)ₚ(1-4C)alkyl
(104) R⁹ is selected from amino(1-4C)alkyl, (1-4C)alkylamino(1-4C)alkyl and di(1-4C)alkylamino(1-4C)alkyl
(105) R⁹ is selected from (1-4C)alkylcarbonylamino, (1-4C)alkylcarbonyl-N-[(1-4C)alkyl]amino, di(1-4C)alkylaminocarbonyl and (1-4C)alkylaminocarbonyl
(106) R¹⁰ is methoxy
(107) R¹⁰ is methyl
(108) R¹⁰ is halo
(109) one R⁹ or R¹⁰ is halo and the other is absent
(110) m is 0
(111) m is 1
(112) n is 0
(113) n is 1
(114) n is 2
(115) m is 1 and n is 0
(116) R¹¹ is hydrogen
(117) R¹¹ is (1-4C)alkyl, such as methyl

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —C(O)NR⁴R⁵;

R³ is halo, methoxy or cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁵ is hydrogen or (1-4C)alkyl;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹ is halo, methyl or methoxy;
R¹⁰ is absent;
R¹¹ is hydrogen;
m is 0 or 1;
n is 0 or 1.

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —C(O)NR⁴R⁵;

R³ is halo, methoxy or cyano;
R⁴ is selected from (1-4C)alkyl;
R⁵ is hydrogen or (1-4C)alkyl;
R⁹ is halo, methyl or methoxy;
R¹⁰ is absent;
R¹¹ is hydrogen;
m is 0 or 1;
n is 0 or 1.

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —C(O)NR⁴R⁵;

R³ is halo, methoxy or cyano;
R⁴ and R⁵ together with the nitrogen atom to which they are attached may form a heterocyclyl ring system as defined by HET-3;
HET-3 is an azetidine, pyrrolidine or piperidine ring, and is optionally substituted by methoxy, hydroxy or methyl;
R⁹, where present, is halo, methyl or methoxy;
R¹⁰ is absent;
R¹¹ is hydrogen;
m is 0 or 1;
n is 0 or 1.

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —SO₂NR⁴R⁵;

R³ is halo, methoxy or cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁵ is hydrogen or (1-4C)alkyl;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹, where present, is halo, methyl or methoxy;
R¹⁰ is absent;
R¹¹ is hydrogen;
m is 0 or 1;
n is 0 or 1;
HET-1 and HET-2 are as hereinbefore defined.

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —SO₂NR⁴R⁵;

R³ is halo, methoxy or cyano;
R⁴ is selected from (1-4C)alkyl;
R⁵ is hydrogen or (1-4C)alkyl;
R⁹, where present, is halo, methyl or methoxy;
R¹⁰ is absent;
R¹¹ is hydrogen;
m is 0 or 1;
n is 0 or 1;
HET-1 is as hereinbefore defined.

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —SO₂NR⁴R⁵;

R³ is halo, methoxy or cyano;
R⁴ and R⁵ together with the nitrogen atom to which they are attached may form a heterocyclyl ring system as defined by HET-3;
HET-3 is an azetidine, pyrrolidine or piperidine ring, and is optionally substituted by methoxy, hydroxy or methyl;
R⁹ is halo, methyl or methoxy;
R¹⁰ is absent;
R¹¹ is hydrogen;
m is 0 or 1;
n is 0 or 1;
HET-1 is as hereinbefore defined.

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —S(O)ₚR⁴;

R³ is halo, methoxy or cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷) and —C(O)NR⁵R⁵], (3-6C)cycloalkyl (optionally substituted with 1 group selected from R⁷) and HET-2;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹, where present, is halo, methyl or methoxy;
R¹⁰ is absent;
R¹¹ is hydrogen;
p is independently at each occurrence 0, 1 or 2;
m is 0 or 1;
n is 0 or 1;
HET-1 and HET-2 are as hereinbefore defined.

In a further aspect of the invention is provided a compound of the formula (I), (IA), (IB), and/or (IC) as hereinbefore defined or a salt thereof wherein:

Ring A is selected from phenyl and HET-1;

### L is —O— or —CH₂O—;

R¹ is (1-6C)alkyl, optionally substituted by a substituent selected from hydroxy and (1-4C)alkoxy;

### R² is —S(O)ₚR⁴;

R³ is halo, methoxy or cyano;

### R⁴ is (1-4C)alkyl;

R⁹ is halo, methyl or methoxy;
R¹⁰ is absent;
R¹¹ is hydrogen;
p is independently at each occurrence 0, 1 or 2;
m is 0 or 1;
n is 0 or 1;
HET-1 is as hereinbefore defined.

In a further aspect of the invention is provided a compound of the formula (I), (IA), (IB), and/or (IC) as hereinbefore defined or a salt thereof wherein:

Ring A is selected from phenyl and HET-1;

### L is —O— or -(1-3C)alkylO—;

R¹ is (1-6C)alkyl, optionally substituted by a substituent selected from hydroxy and (1-4C)alkoxy;

### R² is HET-2;

R³ is halo, methoxy or cyano;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available nitrogen atom by a substituent selected from R⁶ and/or on an available carbon atom by 1 or 2 substituents independently selected from R⁷;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁹, where present, is halo, methyl or methoxy;
R¹⁰ is absent;
R¹¹ is hydrogen;
p is independently at each occurrence 0, 1 or 2;
m is 0 or 1;
n is 0 or 1
HET-1 is as hereinbefore defined.

In a further aspect of the invention is provided a compound of the formula (I), (IA), (IB), and/or (IC) as hereinbefore defined or a salt thereof wherein:

Ring A is phenyl;

### L is —O— or -(1-3C)alkylO—;

R¹ is (1-6C)alkyl, optionally substituted by a substituent selected from hydroxy and (1-4C)alkoxy;
R² is selected from methylsulfonyl, ethylsulfonyl, methylsulfinyl, azetidinylcarbonyl, pyrrolidinylmethyl, dimethylaminocarbonyl, and oxadiazolyl;
R³ is selected from fluoro, chloro, cyano, methoxy and carboxy;
R⁹, where present, is halo, methyl or methoxy;
R¹⁰ is absent;
R¹¹ is hydrogen;
m is 0 or 1;
n is 0 or 1.

In a further aspect of the invention is provided a compound of the formula (I), (IA), (IB), and/or (IC) as hereinbefore defined or a salt thereof wherein:

Ring A is phenyl or pyrimidinyl, particularly phenyl;

### L is —O— or -(1-3C)alkylO—;

R¹ is (1-6C)alkyl, optionally substituted by a substituent selected from hydroxy and (1-4C)alkoxy;
R² is selected from methylsulfonyl and azetidinylcarbonyl;
R³ is selected from fluoro, chloro, cyano, methoxy and carboxy;
R⁹, where present, is halo, methyl or methoxy;
R¹⁰ is absent;
R¹¹ is hydrogen;
m is 0 or 1;
n is 0 or 1.

Further preferred compounds of the invention are each of the Examples, each of which provides a further independent aspect of the invention. In further aspects, the present invention also comprises any two or more compounds of the Examples.

In one aspect, particular compounds of the invention comprise any one or more of:
2-{3-(benzyloxy)-5-[(1S)-2-methoxy-1-methylethoxy]phenyl}-1H-pyrrolo[2,3-b]pyridine;
2-{3-[(1S)-2-methoxy-1-methylethoxy]-5-[4-(methylsulfonyl)phenoxy]phenyl}-1H-pyrrolo[2,3-b]pyridine;
2-{3-{[2-(azetidin-1-ylcarbonyl)pyrimidin-5-yl]oxy}-5-[(1S)-2-methoxy-1-methylethoxy]phenyl}-1H-pyrrolo[2,3-b]pyridine; and/or
8-[3-[(2S)-1-methoxypropan-2-yl]oxy-5-phenylmethoxy-phenyl]-2,5,7-triazabicyclo[4.3.0]nona-2,4,8,10-tetraene;
   or a salt thereof.

The compounds of the invention may be administered in the form of a pro-drug. A pro-drug is a bioprecursor or pharmaceutically acceptable compound being degradable in the body to produce a compound of the invention (such as an ester or amide of a compound of the invention, particularly an in-vivo hydrolysable ester). Various forms of prodrugs are known in the art. For examples of such prodrug derivatives, see:

a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen;

### c) H. Bundgaard, Chapter 5 “Design and Application of Prodrugs”, by H. Bundgaard p. 113-191 (1991);

### d) H. Bundgaard, Advanced Drug Delivery Reviews, 8, 1-38 (1992);

### e) H. Bundgaard, et al., Journal of Pharmaceutical Sciences, 77, 285 (1988); and

### f) N. Kakeya, et al., Chem Pharm Bull, 32, 692 (1984).

The contents of the above cited documents are incorporated herein by reference.

Examples of pro-drugs are as follows. An in-vivo hydrolysable ester of a compound of the invention containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁ to C₆alkoxymethyl esters for example methoxymethyl, C₁ to C₆alkanoyloxymethyl esters for example pivaloyloxymethyl, phthalidyl esters, C₃ to C₈cycloalkoxycarbonyloxyC₁ to C₆alkyl esters for example 1-cyclohexylcarbonyloxyethyl; 1,3-dioxolen-2-onylmethyl esters, for example 5-methyl-1,3-dioxolen-2-onylmethyl; and (1-6C)alkoxycarbonyloxyethyl esters.

An in-vivo hydrolysable ester of a compound of the invention containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a benzoxazinone derivative of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

A further feature of the invention is a pharmaceutical composition comprising a compound of Formula (I), (IA), (IB) or (IC) as defined above, or a pharmaceutically-acceptable salt thereof, together with a pharmaceutically-acceptable diluent or carrier.

According to another aspect of the invention there is provided a compound of Formula (I), (IA), (IB) or (IC) as defined above or a pharmaceutically-acceptable salt thereof for use as a medicament.

According to another aspect of the invention there is provided a compound of Formula (I), (IA), (IB) or (IC) as defined above or a pharmaceutically-acceptable salt thereof for use as a medicament for treatment or prevention, particularly treatment of diabetes and/pr obesity, in particular type 2 diabetes.

According to another aspect of the invention there is provided the use of a compound of Formula (I), (IA), (IB) or (IC) or a pharmaceutically-acceptable salt thereof, in the preparation of a medicament for use in the treatment of diabetes and/or obesity.

Further according to the invention there is provided a compound of Formula (I), (IA), (IB) or (IC) or a pharmaceutically-acceptable salt thereof for use in the preparation of a medicament for treatment of a disease mediated through GLK, in particular type 2 diabetes.

The compound is suitably formulated as a pharmaceutical composition for use in this way.

According to another aspect of the present invention there is provided a method of treating GLK mediated diseases, especially diabetes, by administering an effective amount of a compound of Formula (I), (IA), (IB) or (IC) or a pharmaceutically-acceptable salt thereof, to a mammal in need of such treatment.

Specific diseases which may be treated by a compound or composition of the invention include: blood glucose lowering in Type 2 Diabetes Mellitus without a serious risk of hypoglycaemia (and potential to treat type 1), dyslipidemia, obesity, insulin resistance, metabolic syndrome X, impaired glucose tolerance.

As discussed above, thus the GLK/GLKRP system can be described as a potential “Diabesity” target (of benefit in both Diabetes and Obesity). Thus, according to another aspect of the invention there is provided the use of a compound of Formula (I), (IA), (IB) or (IC) or a pharmaceutically-acceptable salt thereof, in the preparation of a medicament for use in the combined treatment or prevention of diabetes and obesity.

According to another aspect of the invention there if provided the use of a compound of Formula (I), (IA), (IB) or (IC) or a pharmaceutically-acceptable salt thereof, in the preparation of a medicament for use in the treatment or prevention of obesity.

According to a further aspect of the invention there is provided a method for the combined treatment of obesity and diabetes by administering an effective amount of a compound of Formula (I), (IA), (IB) or (IC) or a pharmaceutically-acceptable salt thereof, to a mammal in need of such treatment.

According to a further aspect of the invention there is provided a method for the treatment of obesity by administering an effective amount of a compound of Formula (I), (IA), (IB) or (IC) or a pharmaceutically-acceptable salt thereof, to a mammal in need of such treatment.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). Dosage forms suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound of the Formula (I), (IA), (IB) or (IC) will naturally vary according to the nature and severity of the conditions, the age and sex of the animal or patient and the route of administration, according to well known principles of medicine.

In using a compound of the Formula (I), (IA), (IB) or (IC) for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

The elevation of GLK activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example in the treatment of diabetes mellitus, chemotherapy may include the following main categories of treatment:

1) Insulin and insulin analogues;
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1,6 bisphosphatase inhibitors, glycogen phopsphorylase inhibitors, glycogen synthase kinase inhibitors);
6) Agents designed to reduce the absorption of glucose from the intestine (for example acarbose);
7) Agents that prevent the reabsorption of glucose by the kidney (SGLT inhibitors);
8) Agents designed to treat the complications of prolonged hyperglycaemia (for example aldose reductase inhibitors);
9) Anti-obesity agents (for example sibutramine and orlistat);
10) Anti-dyslipidaemia agents such as, HMG-CoA reductase inhibitors (eg statins); PPARα agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
11) Antihypertensive agents such as, β blockers (eg atenolol, inderal); ACE inhibitors (eg lisinopril); Calcium antagonists (eg. nifedipine); Angiotensin receptor antagonists (eg candesartan), a antagonists and diuretic agents (eg. furosemide, benzthiazide);
12) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
13) Agents which antagonise the actions of glucagon; and
14) Anti-inflammatory agents, such as non-steroidal anti-inflammatory drugs (eg. aspirin) and steroidal anti-inflammatory agents (eg. cortisone).

According to another aspect of the present invention there is provided individual compounds produced as end products in the Examples set out below and salts thereof.

A compound of the invention, or a salt thereof, may be prepared by any process known to be applicable to the preparation of such compounds or structurally related compounds. Functional groups may be protected and deprotected using conventional methods. For examples of protecting groups such as amino and carboxylic acid protecting groups (as well as means of formation and eventual deprotection), see T. W. Greene and P. G. M. Wuts, “Protective Groups in Organic Synthesis”, Second Edition, John Wiley & Sons, New York, 1991.

Processes for the synthesis of compounds of Formula (I), (IA), (IB) or (IC) are provided as a further feature of the invention. Thus, according to a further aspect of the invention there is provided a process for the preparation of a compound of Formula (I), which comprises a process a) to h) (wherein the variables are as defined hereinbefore for compounds of Formula (I) unless otherwise defined):

(a) reaction of a compound of Formula (III) with a compound of Formula (IV),

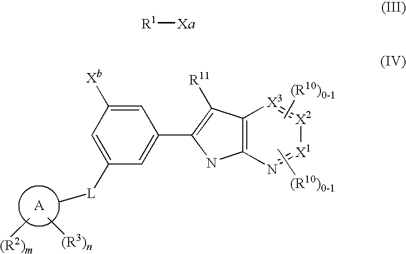

wherein X^{a} is a leaving group or an organometallic reagent and X^{b} is a hydroxyl group, or X^{a} is a hydroxyl group and X^{b} is a leaving group or an organometallic reagent, and wherein R¹ is as defined for a compound of formula (I), or is a protected version thereof; or
(b) reaction of a compound of Formula (V) with a compound of Formula (VI)

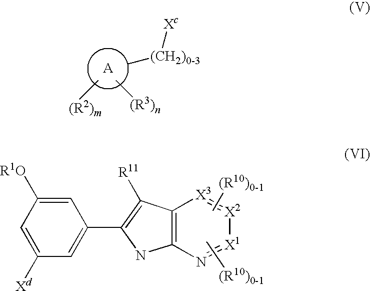

wherein X^{c} is a leaving group or an organometallic reagent and X^{d} is a hydroxyl group, or X^{c} is a hydroxyl group and X^{d} is a leaving group or an organometallic reagent, and wherein R¹ is as defined for a compound of formula (I), or is a protected version thereof; or
(c) reaction of a compound of Formula (VII) with a compound of Formula (VIII),

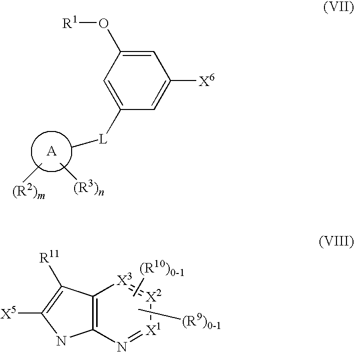

wherein X⁵ is a leaving group and X⁶ is an metal, or X⁶ is a leaving group and X⁵ is an metal; and wherein R¹ is as defined for a compound of formula (I) or is a protected version thereof; or
(d) reaction of a compound of formula (IX) with a compound of formula (X) and cyclisation in a one or two step reaction;

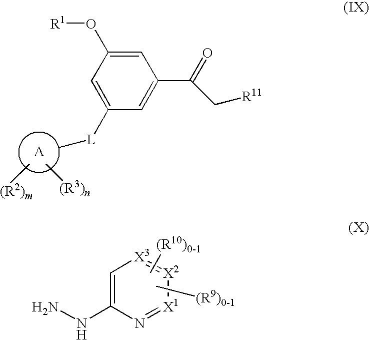

wherein R¹ and R¹¹ are as defined for a compound of formula (I) or a protected version thereof; or
e) reaction of a compound of formula (XI) with a compound of formula (XII) followed by cyclisation,

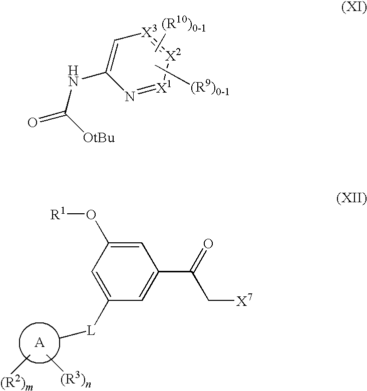

wherein X⁷ is a halogen, or other leaving group, such as —OR (wherein —OR represents an ester or activated ester), and wherein R¹ is as defined for a compound of formula (I) or is a protected version thereof; or
f) reaction of a compound of formula (XIII) with a compound of formula (XIV) followed by cyclisation,

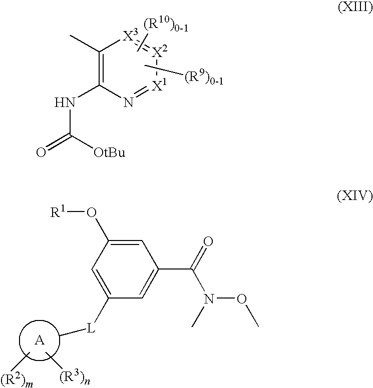

wherein R¹ is as defined for a compound of formula (I) or is a protected version thereof; or
g) reaction of a compound of formula (XV) with a compound of formula (XVI) in the presence of strong base;

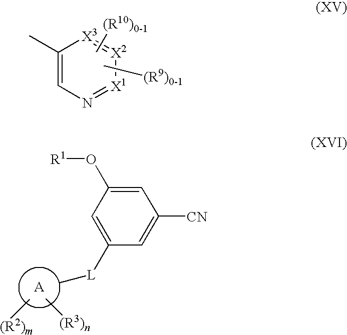

or
h) reaction of a compound of formula (XVII) with a compound of formula (XVIII) in the presence of strong base:

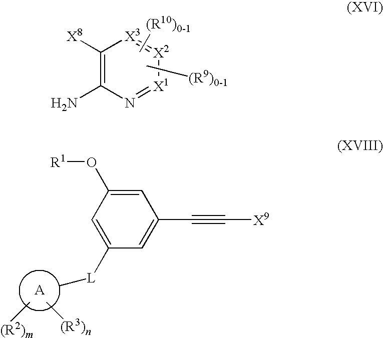

wherein X⁸ is halogen or other suitable leaving group and X⁹ is trimethyl silyl or R¹¹ (where R¹¹ is as defined for a compound of formula (I)), and R¹ is as defined for a compound of formula (I) or a protected version thereof;
and thereafter, if necessary:
i) converting a compound of Formula (I) into another compound of Formula (I);
ii) removing any protecting groups; and/or
iii) forming a salt thereof.

Suitable leaving groups X^{a}—X^{d} and X⁵ to X⁸ for processes a) to h) are any leaving groups known in the art for these types of reactions, for example halo, alkoxy, trifluoromethanesulfonyloxy, methanesulfonyloxy, p-toluenesulfonyloxy, or an organometallic moiety; or a group (such as a hydroxy group) that may be converted in situ into a leaving group (such as an oxytriphenylphosphonium group).

Compounds of Formulae (III) to (XVIII) are commercially available, or are known in the art, or may be made by processes known in the art, for example as shown in the accompanying Examples. For further information on processes for making such compounds, we refer to our PCT publications WO 03/000267, WO 03/015774, WO 03/000262, WO2005/080359 and WO2005/080360 and references therein.

Examples of conversions of a compound of Formula (I) into another compound of Formula (I), well known to those skilled in the art, include functional group interconversions such as hydrolysis, hydrogenation, hydrogenolysis, oxidation or reduction, and/or further functionalisation by standard reactions such as amide or metal-catalysed coupling, or nucleophilic displacement reactions;

Specific reaction conditions for the above reactions are as follows:

Processes a and b)—nucleophilic substitution reactions of alcohols or phenols (or, preferably, their anionic forms) with a suitable electrophile are well known in the art. For example,
(i) using an appropriate substitution reaction, such as an alkoxide with an aryl halide or triflate in a suitable solvent such as dimethylformamide (DMF), dimethylacetamide (DMA), N-methylpyrrolidone (NMP), or dimethyl sulfoxide (DMSO), at a temperature in the range 0 to 200° C., optionally using microwave heating, and optionally using metal catalysis such as palladium (II), palladium (0), copper (II) or copper (I); or
(ii) using an appropriate substitution reaction, such as a phenoxide with an alkyl halide or triflate in a suitable solvent such as dimethylformamide (DMF), dimethylacetamide (DMA), N-methylpyrrolidone (NMP), or dimethyl sulfoxide (DMSO), at a temperature in the range 0 to 200° C., optionally using microwave heating, and optionally using metal catalysis such as palladium (II), palladium (0), copper (II) or copper (I); or
Process c)—suitable metals are boron, tin, zinc and magnesium. Suitable leaving groups are halo and trifluoromethanesulfonate; compounds of Formula (VII) and (VIII) can be reacted together in a suitable solvent, such as DMF, THF or toluene, with a base such as sodium carbonate, potassium carbonate, or potassium tert-butoxide, at a temperature in the range 0 to 200° C., optionally using microwave heating or metal catalysis such as palladium(II), palladium(0), copper(II) or copper(I);
Process d)—Compounds of Formula (IX) and Formula (X) can be reacted together in a suitable solvent, such as ethanol, toluene or acetic acid, with an acid catalyst such as zinc chloride, phosphoric acid, p-toluene sulfonic acid or sulfuric acid, at a temperature in the range 0 to 200° C., optionally using microwave heating, in a one or two step reaction. The reaction may also be performed thermally without addition of a catalyst, optionally using microwave heating.
Process d) is well known in the art as the Fischer Indole Synthesis (see for example ‘The Fischer Indole Synthesis’, Robinson, B, John Wiley and Sons, Chichester, N.Y., 1982).
Process e)—reaction of a compound of Formula (XI) with a compound of Formula (XII) is carried out using n-butyl lithium, t-butyl lithium or another suitable base. Dehydration of the resulting intermediate is carried out using an appropriate acid, such as trifluoroacetic acid (TFA) or hydrochloric acid, optionally in the presence of a suitable solvent and at a temperature of 0-200° C.
Process f)—reaction of a compound of Formula (XIII) with a compound of Formula (XIV) is carried out using n-butyl lithium or another suitable base. Dehydration of the resulting intermediate is carried out using an appropriate acid, such as trifluoroacetic acid (TFA) or hydrochloric acid, optionally in the presence of a suitable solvent and at a temperature of 0-200° C., as described in Synthesis 1996, p 877.
Process g)—reaction of a compound of Formula (XV) with a compound of Formula (XVI) is carried out using n-butyl lithium or another suitable base, in a solvent such as tetrahydrofuran (THF) and at a temperature of −78° C. to 70° C., as described in EP1388541 and WO 03/000688.
Process h)—Suitable leaving groups are halo and trifluoromethanesulfonate; compounds of Formula (XVII) and (XVIII) can be reacted together in a suitable solvent, such as DMF, THF or toluene, with a base such as sodium carbonate, potassium carbonate, or potassium tert-butoxide, at a temperature in the range 0 to 200° C., using metal catalysis such as palladium(II), palladium(0), copper(II) or copper(I), optionally using microwave heating, as described in Tetrahedron Letters, 38(7) pp 627-630 (1998), and WO 03/000688, p 99.

Certain intermediates of formula (III), (IV), (V), (VI), (VII), (VIII), (IX), (X), (XI), (XII), (XIII), (XIV), (XV), (XVI), (XVII) and/or (XVIII) are believed to be novel and comprise an independent aspect of the invention.

During the preparation process, it may be advantageous to use a protecting group for a functional group within the molecule. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower” signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned is of course within the scope of the invention.

A carboxy protecting group may be the residue of an ester-forming aliphatic or araliphatic alcohol or of an ester-forming silanol (the said alcohol or silanol preferably containing 1-20 carbon atoms). Examples of carboxy protecting groups include straight or branched chain (1-12C)alkyl groups (e.g. isopropyl, t-butyl); lower alkoxy lower alkyl groups (e.g. methoxymethyl, ethoxymethyl, isobutoxymethyl; lower aliphatic acyloxy lower alkyl groups, (e.g. acetoxymethyl, propionyloxymethyl, butyryloxymethyl, pivaloyloxymethyl); lower alkoxycarbonyloxy lower alkyl groups (e.g. 1-methoxycarbonyloxyethyl, 1-ethoxycarbonyloxyethyl); aryl lower alkyl groups (e.g. p-methoxybenzyl, o-nitrobenzyl, p-nitrobenzyl, benzhydryl and phthalidyl); tri(lower alkyl)silyl groups (e.g. trimethylsilyl and t-butyldimethylsilyl); tri(lower alkyl)silyl lower alkyl groups (e.g. trimethylsilylethyl); and (2-6C)alkenyl groups (e.g. allyl and vinylethyl).

Methods particularly appropriate for the removal of carboxyl protecting groups include for example acid-, metal- or enzymically-catalysed hydrolysis.

Examples of hydroxy protecting groups include methyl, t-butyl, lower alkenyl groups (e.g. allyl); lower alkanoyl groups (e.g. acetyl); lower alkoxycarbonyl groups (e.g. t-butoxycarbonyl); lower alkenyloxycarbonyl groups (e.g. allyloxycarbonyl); aryl lower alkoxycarbonyl groups (e.g. benzoyloxycarbonyl, p-methoxybenzyloxycarbonyl, o-nitrobenzyloxycarbonyl, p-nitrobenzyloxycarbonyl); tri lower alkyl/arylsilyl groups (e.g. trimethylsilyl, t-butyldimethylsilyl, t-butyldiphenylsilyl); tetrahydropyran-2-yl; aryl lower alkyl groups (e.g. benzyl) groups; and triaryl lower alkyl groups (e.g. triphenylmethyl).

Examples of amino protecting groups include formyl, aralkyl groups (e.g. benzyl and substituted benzyl, e.g. p-methoxybenzyl, nitrobenzyl and 2,4-dimethoxybenzyl, and triphenylmethyl); di-p-anisylmethyl and furylmethyl groups; lower alkoxycarbonyl (e.g. t-butoxycarbonyl); lower alkenyloxycarbonyl (e.g. allyloxycarbonyl); aryl lower alkoxycarbonyl groups (e.g. benzyloxycarbonyl, p-methoxybenzyloxycarbonyl, o-nitrobenzyloxycarbonyl, p-nitrobenzyloxycarbonyl; trialkylsilyl (e.g. trimethylsilyl and t-butyldimethylsilyl); alkylidene (e.g. methylidene); benzylidene and substituted benzylidene groups.

Methods appropriate for removal of hydroxy and amino protecting groups include, for example, nucleophilic displacement, acid-, base, metal- or enzymically-catalysed hydrolysis, or photolytically for groups such as o-nitrobenzyloxycarbonyl, or with fluoride ions for silyl groups. For example, methylether protecting groups for hydroxy groups may be removed by trimethylsilyliodide. A tert-butyl ether protecting group for a hydroxy group may be removed by hydrolysis, for example by use of hydrochloric acid in methanol.

Examples of protecting groups for amide groups include aralkoxymethyl (e.g. benzyloxymethyl and substituted benzyloxymethyl); alkoxymethyl (e.g. methoxymethyl and trimethylsilylethoxymethyl); tri alkyl/arylsilyl (e.g. trimethylsilyl, t-butyldimethylsily, t-butyldiphenylsilyl); tri alkyl/arylsilyloxymethyl (e.g. t-butyldimethylsilyloxymethyl, t-butyldiphenylsilyloxymethyl); 4-alkoxyphenyl (e.g. 4-methoxyphenyl); 2,4-di(alkoxy)phenyl (e.g. 2,4-dimethoxyphenyl); 4-alkoxybenzyl (e.g. 4-methoxybenzyl); 2,4-di(alkoxy)benzyl (e.g. 2,4-di(methoxy)benzyl); and alk-1-enyl (e.g. allyl, but-1-enyl and substituted vinyl e.g. 2-phenylvinyl).

Aralkoxymethyl groups may be introduced onto the amide group by reacting the latter group with the appropriate aralkoxymethyl chloride, and removed by catalytic hydrogenation. Alkoxymethyl, tri alkyl/arylsilyl and tri alkyl/silyloxymethyl groups may be introduced by reacting the amide with the appropriate chloride and removing with acid; or in the case of the silyl containing groups, fluoride ions. The alkoxyphenyl and alkoxybenzyl groups are conveniently introduced by arylation or alkylation with an appropriate halide and removed by oxidation with ceric ammonium nitrate. Finally alk-1-enyl groups may be introduced by reacting the amide with the appropriate aldehyde and removed with acid.

In the above other pharmaceutical composition, process, method, use and medicament manufacture features, the alternative and preferred aspects and embodiments of the compounds of the invention described herein also apply.

The following examples are for illustration purposes and are not intended to limit the scope of this application. Each exemplified compound represents a particular and independent aspect of the invention. In the following non-limiting Examples, unless otherwise stated:

(i) evaporations were carried out by rotary evaporation in vacuo and work-up procedures were carried out after removal of residual solids such as drying agents by filtration;

(ii) operations were carried out at room temperature, that is in the range 18-25° C. and under an atmosphere of an inert gas such as argon or nitrogen;

(iii) yields are given for illustration only and are not necessarily the maximum attainable;

(iv) the structures of the end-products of the Formula (I) were confirmed by nuclear (generally proton) magnetic resonance (NMR) with a field strength (for proton) of 300 MHz (generally using a Varian Gemini 2000) or 400 MHz (generally using a Bruker Avance DPX400), unless otherwise stated, and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;

(v) intermediates were not generally fully characterised and purity was assessed by thin layer chromatography (TLC), high-performance liquid chromatography (HPLC), infra-red (IR) or NMR analysis; and

(vi) Purification by chromatography generally refers to flash column chromatography, on silica unless otherwise stated. Column chromatography was generally carried out using prepacked silica cartridges (from 4 g up to 400 g) such as Redisep™ (available, for example, from Presearch Ltd, Hitchin, Herts, UK) or Biotage (Biotage UK Ltd, Hertford, Herts, UK), eluted using a pump and fraction collector system;

(vii) Mass spectra (MS) data was generated on an LCMS system where the HPLC component comprised generally either a Agilent 1100 or Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water:acetonitrile (v/v) mixture; or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ spectrometer. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is (M+H)+ for positive ion mode and (M−H)⁻ for negative ion mode;

(viii) Suitable microwave reactors include “Smith Creator”, “CEM Explorer”, “Biotage Initiator sixty” and “Biotage Initiator eight”.

(ix) Preparative HPLC separations were run on standard Gilson™ HPLC equipment using a 150×21.2 mm Phenomenex Luna 10 micron C18(2) 100A column, and a standard gradient elution method (5-95% acetonitrile gradient with water as co-solvent and 0.2% trifluoroacetic acid as modifier, 12.5 min gradient with a 2.5 min hold at 95% acetonitrile) run on Unipoint software.

### Abbreviations:

DCM dichloromethane
DMA dimethylacetamide
MgSO₄ magnesium sulfate
THF tetrahydrofuran
DMSO dimethylsulfoxide
MeOH methanol
MeOD per-deuteromethanol
DMF dimethylformamide
TFA trifluoroacetic acid
LDA lithium diisopropylamide
EDAC N-ethyl-N′-(3-dimethylaminopropyl)-carbodiimide
DMAP 4-dimethylamino pyridine
EtOAc ethyl acetate
All final compound names were derived using ACD NAME computer package.

### EXAMPLE 1

### 2-[3-(Benzyloxy)-5-[(1S)-2-methoxy-1-methylethoxy]phenyl]-1H-pyrrolo[2,3-b]pyridine

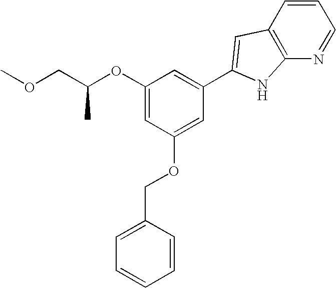

n-Butyl lithium (2.6 mL of a 1.6M solution in hexanes, 4.2 mmol) was added dropwise to a solution of tert-butyl (3-methylpyridin-2-yl)carbamate (290 mg, 1.39 mmol) in THF between 0 and −5° C., under argon; the solution turned deep red and was allowed to stir for approx 20 mins. The Weinreb amide (3-(benzyloxy)-N-methoxy-5-[(1S)-2-methoxy-1-methylethoxy]-N-methylbenzamide, 500 mg, 1.39 mmol) was added dropwise as a solution in THF (1 mL). The reaction mixture was then allowed to come to room temperature over approximately 1.5 h. The reaction mixture was quenched with water at 0° C., allowed to come to room temperature and then extracted with ethyl acetate (×3). The combined extracts were washed with brine (×2), dried (MgSO₄) and concentrated in vacuo to yield the crude intermediate as a yellow oil (725 mg) which was reacted without purification or characterisation.

TFA (10 mL) was added to a solution of the crude intermediate from the previous step in DCM (15 mL) at ambient temperature, and the reaction mixture allowed to stir overnight. The reaction mixture was concentrated in vacuo and purified by preparative HPLC to give the title compound (150 mg) as a yellow solid, ¹H NMR (400 MHz, CDCl₃) δ 1.40 (d, 3H), 3.49 (s, 3H), 3.63 (m, 2H), 4.74 (m, 1H), 5.22 (s, 2H), 6.70 (dd, 1H), 6.95 (s, 1H), 7.12 (d, 2H), 7.35 (dd, 2H), 7.42 (dd, 2H), 7.51 (d, 2H), 8.14 (d, 1H), 8.35 (d, 1H), 14.94 (s, 1H), m/z 389, (M+H)⁺.

The requisite (3-(benzyloxy)-N-methoxy-5-[(1S)-2-methoxy-1-methylethoxy]-N-methylbenzamide starting material was prepared as follows:

### (3-(benzyloxy)-N-methoxy-5-[(1S)-2-methoxy-1-methylethoxy]-N-methylbenzamide

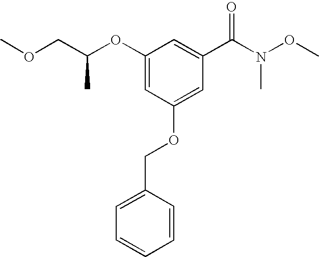

A solution of 3-(benzyloxy)-5-[(1S)-2-methoxy-1-methylethoxy]benzoic acid (10.0 g, 31.6 mmol), N,O-dimethyl hydroxylamine hydrochloride (4.63 g, 47.5 mmol) and DMAP (7.73 g, 63.3 mmol) in DMF (100 mL) was treated with EDAC (9.07 g, 47.5 mmol), and the resulting solution was stirred at ambient temperature overnight. It was then diluted with EtOAc, washed sequentially with water (twice), 1M citric acid, water and brine, dried (MgSO₄) and evaporated to give the crude product as an oil (approximately 10 g). This was purified by chromatography (330 g silica column, CombiFlash™, eluting with hexane containing increasing amounts of EtOAc [25%, 37.5%, 50%]) to give the title compound as a colourless oil, (8.5 g), ¹H NMR (400 MHz, d⁶-DMSO) δ 1.19 (d, 3H), 3.22 (s, 3H), 3.28 (s, 3H), 3.39-3.51 (m, 2H), 3.53 (s, 3H), 4.59-4.69 (m, 1H), 5.12 (s, 2H), 6.65-6.71 (m, 2H), 6.73 (s, 1H), 7.29-7.48 (m, 5H), m/z 358 (M−H)⁻, 360 (M+H)⁺.

The requisite 3-(benzyloxy)-5-[(1S)-2-methoxy-1-methylethoxy]benzoic acid starting material was prepared as described in WO 2005/121110.

### EXAMPLE 2

### 2-{3-[(1S)-2-Methoxy-1-methylethoxy]-5-[4-(methylsulfonyl)phenoxy]phenyl}-1H-pyrrolo[2,3-b]pyridine

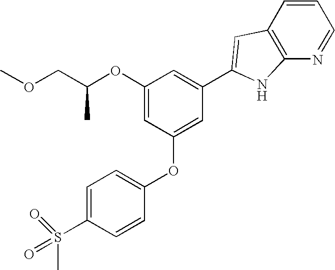

A solution of 3-[(1S)-2-methoxy-1-methylethoxy]-5-(1H-pyrrolo[2,3-b]pyridin-2-yl)phenol (21 mg, 0.07 mmol) in DMA (1 mL) was treated with caesium carbonate (46 mg, 0.14 mmol) and methyl 4-fluorophenyl sulfone (24 mg, 0.14 mmol), and the reaction mixture was heated at 115° C. for approximately 3 h. The reaction mixture was allowed to cool to room temperature, evaporated in vacuo and purified by preparative HPLC to give the title compound (12.4 mg) as a yellow oil, ¹H NMR (400 MHz, MeOD) δ 1.36 (d, 3H), 3.15 (s, 3H), 3.44 (s, 3H), 3.59 (m, 2H), 4.74 (m, 1H), 6.84 (s, 1H), 7.16 (s, 1H), 7.26 (m, 3H), 7.43 (m, 2H), 7.98 (d, 2H), 8.34 (s, 1H), 8.44 (d, 1H), m/z 453 (M+H)⁺.

The requisite phenol starting material was prepared as follows:

### 3-[(1S)-2-Methoxy-1-methylethoxy]-5-(1H-pyrrolo[2,3-b]pyridin-2-yl)phenol

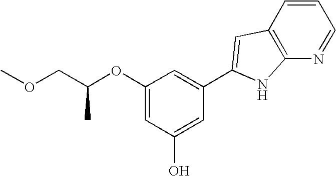

A solution of 2-{3-(benzyloxy)-5-[(1S)-2-methoxy-1-methylethoxy]phenyl}-1H-pyrrolo[2,3-b]pyridine (1.3 g, 3.46 mmol) and ammonium formate (0.437 g, 6.93 mmol) in ethanol (53 mL) was treated with 30% palladium on carbon catalyst, and the mixture was heated with stirring at 80° C. for approximately 2 hrs. The reaction mixture was cooled to room temperature and the catalyst removed by filtration. The filtrate was concentrated in vacuo to yield the title compound as a yellow oil (0.87 g), ¹H NMR (400 MHz, CDCl₃) δ 1.25 (d, 3H), 3.36 (s, 3H), 3.43 (d, 1H), 3.53 (d, 1H), 4.50 (m, 1H), 6.45 (dd, 1H), 6.62 (s, 1H), 6.84 (d, 2H), 7.00 (dd, 1H), 7.85 (dd, 1H), 8.14 (dd, 1H), m/z 299 (M+H)⁺. This was used without further purification.

The requisite 2-{3-(benzyloxy)-5-[(1S)-2-methoxy-1-methylethoxy]phenyl}-1H-pyrrolo[2,3-b]pyridine starting material was prepared as described in Example 1.

### EXAMPLE 3

### 2-{3-{[2-(Azetidin-1-ylcarbonyl)pyrimidin-5-yl]oxy}-5-[(1S)-2-methoxy-1-methylethoxy]phenyl}-1H-pyrrolo[2,3-b]pyridine

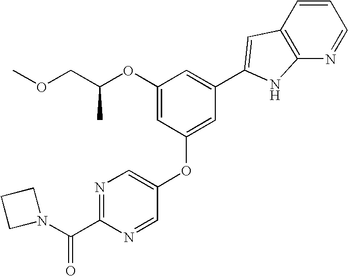

This was prepared in a manner essentially similar to that described in Example 2 starting from 3-[(1S)-2-methoxy-1-methylethoxy]-5-(1H-pyrrolo[2,3-b]pyridin-2-yl)phenol and azetidin-1-yl-(5-bromopyrimidin-2-yl)methanone; the reaction was performed with Microwave heating (200° C., 2 hrs), and with the addition of catalytic bromotris (triphenylphosphine) copper (I). ¹H NMR (400 MHz, CDCl₃) δ 1.29 (d, 3H), 2.33 (s, 2H), 3.34 (s, 3H), 3.50 (m, 2H), 4.25 (s, 2H), 4.55 (s, 2H), 4.67 (m, 2H), 6.67 (s, 1H), 6.87 (s, 1H), 7.05 (s, 1H), 7.26 (t, 1H), 7.31 (s, 1H), 8.08 (s, 1H), 8.28 (d, 2H), 9.05 (s, 1H), 15.17 (s, 1H), m/z 460 (M+H)⁺.

### EXAMPLE 4

### 8-[3-[(2S)-1-Methoxypropan-2-yl]oxy-5-phenylmethoxy-phenyl]-2,5,7-triazabicyclo[4.3.0]nona-2,4,8,10-tetraene

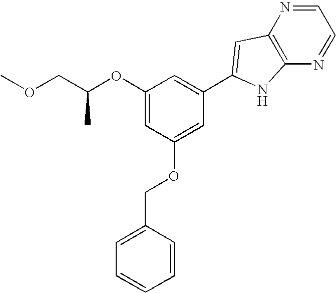

A solution of n-butyl lithium (1.99 mL of a 1.6M solution, 3.19 mmol) was added dropwise to a solution of diisopropylamine (0.447 mL, 3.19 mmol) in THF (10.5 mL), under an argon atmosphere, maintaining the temperature between −16° C. and −11° C. The resulting solution was allowed to stir for approximately 30 mins at this temperature. Methyl pyrazine (0.194 mL, 2.12 mmol) was then added dropwise to the pre-formed LDA solution (held at −15° C.), which instantly turned a deep brown colour. The solution was allowed to stir for a further hour, maintaining the temp between −15° C. and −10° C. A solution of 3-[(2S)-1-methoxypropan-2-yl]oxy-5-phenylmethoxy-benzonitrile (631 mg, 2.12 mmol) in THF (4 mL) was added dropwise, holding the reaction mixture at −15° C. After the addition, the reaction mixture was allowed to warm to ambient temperature over several hours and then left to stand overnight. Water was added to the reaction mixture, the THF was removed in vacuo, and the residue extracted with 4×10 mL portions of ethyl acetate. The organic extracts were then washed with water, dried (MgSO₄) and evaporated in vacuo to give the crude product. This was purified by chromatography (Companion using a 12 g silica column and eluting with DCM containing MeOH, 0-10% gradient) and further purified by preparative HPLC to give the title compound as a bright yellow solid (106 mg). ¹H NMR (400 MHz, MeOD) δ 1.33 (d, 3H), 3.44 (s, 3H), 3.58 (m, 2H), 4.71 (m, 1H), 5.20 (s, 2H), 6.72 (s, 1H), 7.03 (s, 1H), 7.17 (s, 1H), 7.22 (s, 1H), 7.34 (d, 1H), 7.40 (d, 2H), 7.50 (d, 2H), 8.33 (d, 2H), m/z 390 (M+H)⁺.

The requisite 3-[(2S)-1-methoxypropan-2-yl]oxy-5-phenylmethoxy-benzonitrile starting material was prepared as follows:

### 3-[(2S)-1-methoxypropan-2-yl]oxy-5-phenylmethoxy-benzonitrile

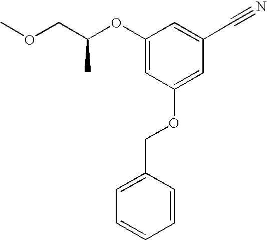

A solution of 3-[(2S)-1-methoxypropan-2-yl]oxy-5-phenylmethoxy-benzamide (6.9 g, 21.9 mmol) and pyridine (7.1 mL, 87.6 mmol) in dioxan was cooled in an ice bath and held just above the freezing point; trifluoroacetic anhydride (TFAA, 7.4 mL, 52.6 mmol) was added over 5 mins, and the reaction mixture stirred a further 15 mins and then allowed to warm to ambient temperature. Stirring was continued for a further 4 hours, then the solvent was removed in vacuo and the residue dissolved in EtOAc (˜200 mL); the resulting solution was washed sequentially with one portion each of water, dilute (IM) citric acid solution and brine, dried (MgSO₄) and evaporated to give the crude product as a colourless oil (7.3 g). This was chromatographed (120 g Si column, Combi-flash Graduate™, eluting with iso-hexane containing EtOAc 0-20%) to give the title compound as a colourless oil (4.3 g). ¹H NMR (400 MHz, d⁶-DMSO) δ 1.20 (d, 3H), 3.28 (s, 3H), 3.43-3.51 (m, 2H), 4.67-4.77 (m, 1H), 5.19 (s, 2H), 6.89-6.95 (m, 1H), 7.01-7.05 (m, 1H), 7.05-7.11 (m, 1H), 7.31-7.49 (m, 5H), m/z 296 (M−H)⁻.

The requisite 3-[(2S)-1-methoxypropan-2-yl]oxy-5-phenylmethoxy-benzamide starting material was prepared as follows:

### 3-[(2S)-1-methoxypropan-2-yl]oxy-5-phenylmethoxy-benzamide

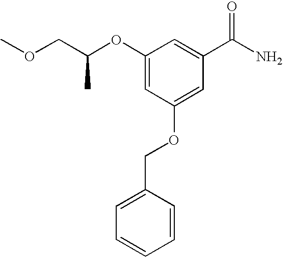

Oxalyl chloride (3.4 ml, 37.9 mmol) was added dropwise to a solution of 3-(benzyloxy)-5-[(1S)-2-methoxy-1-methylethoxy]benzoic acid (10.0 g, 31.6 mmol) in anhydrous DCM (80 mL) containing DMF (1 mL). The solution was stirred under argon at ambient temperature for 30 mins and then concentrated to a yellow oil. The oil was re-dissolved in anhydrous DCM (80 mL) and cooled to 10-15° C. Ammonia (10 mL of a 7M solution in methanol, 69.5 mmol) was added dropwise, and the resulting white suspension was stirred at ambient temperature for 1 hr then concentrated in vacuo. DCM (100 mL) was added and the insoluble material removed by filtration. The filtrate was concentrated to a yellow oil (10.95 g). This was purified by chromatography (120 g silica cartridge, Combiflash Graduate™, eluting with a gradient consisting of hexane containing 25%-75% EtOAc) to give the title compound as a colourless oil, (8.3 g), ¹H NMR (400 MHz, d⁶-DMSO) δ 1.21 (d, 3H), 3.29 (s, 3H), 3.40-3.53 (m, 2H), 4.58-4.71 (m, 1H), 5.13 (s, 2H), 6.72 (s, 1H), 05 (s, 1H), 7.11 (s, 1H), 7.30 (br s, 1H), 7.33-7.50 (m, 5H), 7.89 (br s, 1H); the spectrum also contained peaks due to solvent (ethyl acetate), m/z 316 (M+H)⁺.

The requisite 3-(benzyloxy)-5-[(1S)-2-methoxy-1-methylethoxy]benzoic acid starting material was prepared according to the references given in Example 1.

### Biological

### Tests:

The biological effects of the compounds of formula (I) may be tested in the following way:

### (1) Enzymatic Activity

Enzymatic activity of recombinant human pancreatic GLK may be measured by incubating GLK, ATP and glucose. The rate of product formation may be determined by coupling the assay to a G-6-P dehydrogenase, NADP/NADPH system and measuring the linear increase with time of optical density at 340 nm (Matschinsky et al 1993). Activation of GLK by compounds can be assessed using this assay in the presence or absence of GLKRP as described in Brocklehurst et al (Diabetes 2004, 53, 535-541).

### Production of Recombinant GLK and GLKRP:

Human GLK and GLKRP cDNA was obtained by PCR from human pancreatic and hepatic mRNA respectively, using established techniques described in Sambrook J, Fritsch E F & Maniatis T, 1989. PCR primers were designed according to the GLK and GLKRP cDNA sequences shown in Tanizawa et al 1991 and Bonthron, D. T. et al 1994 (later corrected in Warner, J. P. 1995).

### Cloning in Bluescript II Vectors

GLK and GLKRP cDNA was cloned in E. coli using pBluescript II, (Short et al 1998) a recombinant cloning vector system similar to that employed by Yanisch-Perron C et al (1985), comprising a colEI-based replicon bearing a polylinker DNA fragment containing multiple unique restriction sites, flanked by bacteriophage T3 and T7 promoter sequences; a filamentous phage origin of replication and an ampicillin drug resistance marker gene.

### Transformations

E. Coli transformations were generally carried out by electroporation. 400 mL cultures of strains DH5a or BL21 (DE3) were grown in L-broth to an OD 600 of 0.5 and harvested by centrifugation at 2,000 g. The cells were washed twice in ice-cold deionised water, resuspended in 1 mL 10% glycerol and stored in aliquots at −70° C. Ligation mixes were desalted using Millipore V Series™ membranes (0.0025 mm) pore size). 40 mL of cells were incubated with 1 mL of ligation mix or plasmid DNA on ice for 10 minutes in 0.2 cm electroporation cuvettes, and then pulsed using a Gene Pulser™ apparatus (BioRad) at 0.5 kVcm⁻¹, 250 mF. Transformants were selected on L-agar supplemented with tetracycline at 10 mg/mL or ampicillin at 100 mg/mL.

### Expression

GLK was expressed from the vector pTB375NBSE in E. coli BL21 cells, producing a recombinant protein containing a 6-His tag immediately adjacent to the N-terminal methionine. Alternatively, another suitable vector is pET21 (+)DNA, Novagen, Cat number 697703. The 6-His tag was used to allow purification of the recombinant protein on a column packed with nickel-nitrilotriacetic acid agarose purchased from Qiagen (cat no 30250).

GLKRP was expressed from the vector pFLAG CTC (IBI Kodak) in E. coli BL21 cells, producing a recombinant protein containing a C-terminal FLAG tag. The protein was purified initially by DEAE Sepharose ion exchange followed by utilisation of the FLAG tag for final purification on an M2 anti-FLAG immunoaffinity column purchased from Sigma-Aldrich (cat no. A1205).

Compounds of the invention generally have an activating activity for glucokinase with an EC₅₀ of less than about 30 μM, particularly less than about 10 μM, preferably less than about 1 μM, more preferably less than about 0.1 μM. For example, Example 1 has an EC₅₀ of 0.12 μM.

### REFERENCES

1 Printz, R. L., Magnuson, M. A. and Granner, D. K. (1993) Annual Review of Nutrition 13, 463-96
2 DeFronzo, R. A. (1988) Diabetes 37, 667-87
3 Froguel, P., Zouali, H., Vionnet, N., Velho, G., Vaxillaire, M., Sun, F., Lesage, S., Stoffel, M., Takeda, J. and Passa, P. (1993) New England Journal of Medicine 328, 697-702
4 Bell, G. I., Pilkis, S. J., Weber, I. T. and Polonsky, K. S. (1996) Annual Review of Physiology 58, 171-86
5 Velho, G., Petersen, K. F., Perseghin, G., Hwang, J. H., Rothman, D. L., Pueyo, M. E., Cline, G. W., Froguel, P. and Shulman, G. I. (1996) Journal of Clinical Investigation 98, 1755-61
6 Christesen, H. B., Jacobsen, B. B., Odili, S., Buettger, C., Cuesta-Munoz, A., Hansen, T., Brusgaard, K., Massa, O., Magnuson, M. A., Shiota, C., Matschinsky, F. M. and Barbetti, F. (2002) Diabetes 51, 1240-6
6a Gloyn, A. L., Noordam, K., Willemsen, M. A. A. P., Ellard, S., Lam, W. W. K., Campbell, I. W., Midgley, P., Shiota, C., Buettger, C., Magnuson, M. A., Matschinsky, F. M., and Hattersley, A. T.; Diabetes 52: 2433-2440
7 Glaser, B., Kesavan, P., Heyman, M., Davis, E., Cuesta, A., Buchs, A., Stanley, C. A., Thornton, P. S., Permutt, M. A., Matschinsky, F. M. and Herold, K. C. (1998) New England Journal of Medicine 338, 226-30
8 Caro, J. F., Triester, S., Patel, V. K., Tapscott, E. B., Frazier, N. L. and Dohm, G. L. (1995) Hormone & Metabolic Research 27, 19-22
9 Desai, U. J., Slosberg, E. D., Boettcher, B. R., Caplan, S. L., Fanelli, B., Stephan, Z., Gunther, V. J., Kaleko, M. and Connelly, S. (2001) Diabetes 50, 2287-95
10 Shiota, M., Postic, C., Fujimoto, Y., Jetton, T. L., Dixon, K., Pan, D., Grimsby, J., Grippo, J. F., Magnuson, M. A. and Chemington, A. D. (2001) Diabetes 50, 622-9
11 Ferre, T., Pujol, A., Riu, E., Bosch, F. and Valera, A. (1996) Proceedings of the National Academy of Sciences of the United States of America 93, 7225-30
12 Seoane, J., Barbera, A., Telemaque-Potts, S., Newgard, C. B. and Guinovart, J. J. (1999) Journal of Biological Chemistry 274, 31833-8
13 Moore, M. C., Davis, S. N., Mann, S. L. and Chemington, A. D. (2001) Diabetes Care 24, 1882-7
14 Alvarez, E., Roncero, I., Chowen, J. A., Vazquez, P. and Blazquez, E. (2002) Journal of Neurochemistry 80, 45-53
15 Lynch, R. M., Tompkins, L. S., Brooks, H. L., Dunn-Meynell, A. A. and Levin, B. E. (2000) Diabetes 49, 693-700
16 Roncero, I., Alvarez, E., Vazquez, P. and Blazquez, E. (2000) Journal of Neurochemistry 74, 1848-57
17 Yang, X. J., Kow, L. M., Funabashi, T. and Mobbs, C. V. (1999) Diabetes 48, 1763-1772
18 Schuit, F. C., Huypens, P., Heimberg, H. and Pipeleers, D. G. (2001) Diabetes 50, 1-11
19 Levin, B. E. (2001) International Journal of Obesity 25, supplement 5, S68-S72.
20 Alvarez, E., Roncero, I., Chowen, J. A., Thorens, B. and Blazquez, E. (1996) Journal of Neurochemistry 66, 920-7
21 Mobbs, C. V., Kow, L. M. and Yang, X. J. (2001) American Journal of Physiology—Endocrinology & Metabolism 281, E649-54
22 Levin, B. E., Dunn-Meynell, A. A. and Routh, V. H. (1999) American Journal of Physiology 276, R1223-31
23 Spanswick, D., Smith, M. A., Groppi, V. E., Logan, S. D. and Ashford, M. L. (1997) Nature 390, 521-5
24 Spanswick, D., Smith, M. A., Mirshamsi, S., Routh, V. H. and Ashford, M. L. (2000) Nature Neuroscience 3, 757-8
25 Levin, B. E. and Dunn-Meynell, A. A. (1997) Brain Research 776, 146-53
26 Levin, B. E., Govek, E. K. and Dunn-Meynell, A. A. (1998) Brain Research 808, 317-9
27 Levin, B. E., Brown, K. L. and Dunn-Meynell, A. A. (1996) Brain Research 739, 293-300
28 Rowe, I. C., Boden, P. R. and Ashford, M. L. (1996) Journal of Physiology 497, 365-77
29 Fujimoto, K., Sakata, T., Arase, K., Kurata, K., Okabe, Y. and Shiraishi, T. (1985) Life Sciences 37, 2475-82
30 Kurata, K., Fujimoto, K. and Sakata, T. (1989) Metabolism: Clinical & Experimental 38, 46-51
31 Kurata, K., Fujimoto, K., Sakata, T., Etou, H. and Fukagawa, K. (1986) Physiology & Behavior 37, 615-20
32 Jetton T. L., Liang Y., Pettepher C. C., Zimmerman E. C., Cox F. G., Horvath K., Matschinsky F. M., and Magnuson M. A., J. Biol. Chem., February 1994; 269: 3641-3654
33 Reimann F. and Gribble F. M., Diabetes 2002 51: 2757-2763
34 Cheung A. T., Dayanandan B., Lewis J. T., Korbutt G. S., Rajotte R. V., Bryer-Ash M., Boylan M. O., Wolfe M. M., Kieffer T. J., Science, Vol 290, Issue 5498, 1959-1962, 8 Dec. 2000

1. A compound of Formula (I): 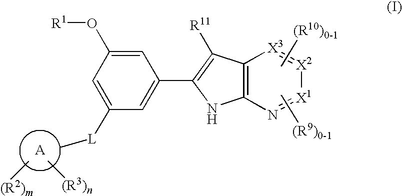
wherein:
Ring A is selected from phenyl and HET-1;
X¹, X² and X³ are each independently CH or N, with the proviso that only one of X¹, X² and X³ may be N;
L is a linker selected from —O— and -(1-3C)alkylO— (wherein the oxygen is directly attached to the central phenyl ring which is substituted by —OR¹);
R¹ is selected from (1-6C)alkyl, (2-6C)alkenyl, (2-6C)alkynyl, (3-6C)cycloalkyl, (3-6C)cycloalkyl(1-6C)alkyl, aryl(1-6C)alkyl, HET-1a and HET-1a-(1-6C)alkyl;
wherein any alkyl, alkenyl, alkynyl, cycloalkyl, aryl or HET-1a group in any definition of;
R¹ may optionally be substituted on an available carbon atom 1 or more halo and/or with a substituent selected from hydroxy, (1-4C)alkoxy, (1-6C)alkylamino, di(1-6C)alkylamino, (C_{q}H_{2q+2-a}Fₐ)—O— (wherein q=1 to 4 and a=1 to 3), (1-6C)alkylsulfonyl, (1-6C)alkylsulfonylamino, (1-6C)alkylsulfonyl-N-[(1-6C)alkyl]amino, (1-6C)alkylaminosulfonyl, di(1-6C)alkylaminosulfonyl, (1-6C)alkylcarbonylamino, (1-6C)alkylcarbonyl-N-[(1-6C)alkyl]amino, (1-6C)alkylaminocarbonyl, di(1-6C)alkylaminocarbonyl, carboxy and cyano; and/or may be substituted on an available nitrogen atom (provided the nitrogen is not thereby quaternised) by a substituent selected from (1-6C)alkylsulfonyl, (1-6C)alkylaminosulfonyl, di(1-6C)alkylaminosulfonyl, (1-6C)alkylaminocarbonyl and di(1-6C)alkylaminocarbonyl;
HET-1 and HET-1a are independently a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group;
R² is selected from —C(O)NR⁴R⁵, —SO₂NR⁴R⁵, —S(O)ₚR⁴ and HET-2;
HET-2 is a 4-, 5- or 6-membered, C- or N-linked saturated, partially or fully unsaturated heterocyclyl ring containing 1, 2, 3 or 4 heteroatoms independently selected from O, N and S, wherein a —CH₂— group can optionally be replaced by a —C(O)—, and wherein a sulphur atom in the heterocyclic ring may optionally be oxidised to a S(O) or S(O)₂ group, which ring is optionally substituted on an available nitrogen atom (provided the nitrogen is not thereby quaternised) by a substituent selected from R⁶ and/or on an available carbon atom by 1 or 2 substituents independently selected from R⁷;
R³ is selected from halo, fluoromethyl, difluoromethyl, trifluoromethyl, methyl, (1-4C)alkoxy, carboxy and cyano;
R⁴ is selected from hydrogen, (1-4C)alkyl [optionally substituted by 1 or 2 substituents independently selected from HET-2, —OR⁵, —SO₂R⁵, (3-6C)cycloalkyl (optionally substituted with 1 group selected from R⁷), cyano, —NR^{4′} R^{5′} and —C(O)NR⁵R⁵], fluoromethyl, difluoromethyl, trifluoromethyl, (3-6C)cycloalkyl (optionally substituted with 1 group selected from R⁷), (2-4C)alkenyl (optionally substituted with 1 group selected from R⁷), (2-4C)alkynyl (optionally substituted with 1 group selected from R⁷), and HET-2;
R⁵ is (independently at each occurrence) selected from hydrogen, (1-4C)alkyl and (3-6C)cycloalkyl;
or R⁴ and R⁵ together with the nitrogen atom to which they are attached may form a heterocyclyl ring system as defined by HET-3;
R^{4′} and R^{5′} are independently selected from hydrogen and (1-4C)alkyl; or
R^{4′} and R^{5′} together with the nitrogen atom to which they are attached may form a 4- to 6-membered saturated ring;
R⁶ is selected from (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
R⁷ is selected from —OR⁵, (1-4C)alkyl, —C(O)(1-4C)alkyl, —C(O)NR⁴R⁵, (1-4C)alkoxy(1-4C)alkyl, hydroxy(1-4C)alkyl and —S(O)pR⁵;
HET-3 is an N-linked, 4 to 7 membered, saturated or partially unsaturated heterocyclyl ring, optionally containing 1 or 2 further heteroatoms (in addition to the linking N atom) independently selected from O, N and S, wherein a —CH₂— group can optionally be replaced by a —C(O)— and wherein a sulphur atom in the ring may optionally be oxidised to a S(O) or S(O)₂ group; which ring is optionally substituted on an available carbon or nitrogen atom by 1 or 2 substituents independently selected from R⁸;
when R⁸ is a substituent on carbon it is selected from halo, —OR⁵, (1-4C)alkyl, (2-4C)alkenyl, (2-4C)alkynyl, trifluoromethyl, —C(O)(1-4C)alkyl, —C(O)NR⁴R⁵, (1-4C)alkylamino, di(1-4C)alkylamino, HET-3 (wherein said ring is unsubstituted), (1-4C)alkoxy(1-4C)alkyl, hydroxy(1-4C)alkyl and —S(O)pR⁵;
when R⁸ is a substituent on nitrogen it is selected from (1-4C)alkyl, —C(O)(1-4C)alkyl, —C(O)NR⁴R⁵, HET-3 (wherein said ring is unsubstituted), (1-4C)alkoxy(2-4C)alkyl, hydroxy(2-4C)alkyl and —S(O)pR⁵;
R⁹ is selected from (1-4C)alkyl, halo, cyano, hydroxy(1-4C)alkyl, dihydroxy(2-4C)alkyl, (1-4C)alkoxy(1-4C)alkyl, di(1-4C)alkoxy(2-4C)alkyl, (1-4C)alkylS(O)ₚ(1-4C)alkyl, amino(1-4C)alkyl, (1-4C)alkylamino(1-4C)alkyl, di(1-4C)alkylamino(1-4C)alkyl, (1-4C)alkylcarbonylamino, (1-4C)alkylcarbonyl-N-[(1-4C)alkyl]amino, (1-4C)alkylaminocarbonyl and di(1-4C)alkylaminocarbonyl;
R¹⁰ is selected from methoxy, methyl and halo;
R¹¹ is selected from hydrogen and (1-4C)alkyl;
p is (independently at each occurrence) 0, 1 or 2;
m is 0 or 1;
n is 0, 1 or 2;
or a salt thereof.

2. A compound as claimed in claim 1, or a salt thereof, wherein R¹¹ is hydrogen.

3. A compound as claimed in claim 1 or claim 2, or a salt thereof, wherein Ring A is selected from phenyl, pyridyl, pyrimidinyl and pyrazinyl.

4. A compound as claimed in any one of claims 1 to 3, or a salt thereof, which is a compound of formula (IA).

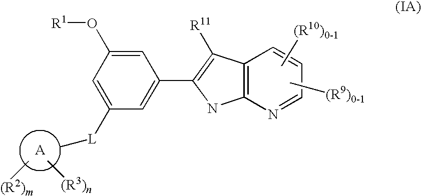

5. A compound as claimed in any one of claims 1 to 3, or a salt thereof, which is a compound of formula (IB).

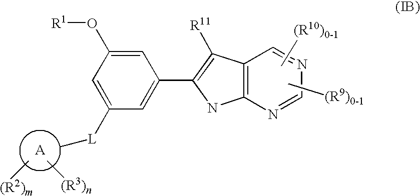

6. A compound as claimed in any one of claims 1 to 3, or a salt thereof, which is a compound of formula (IC);

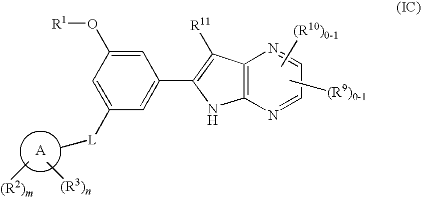

7. A compound of the formula (I) as claimed in claim 1 or a salt thereof wherein:
Ring A is phenyl or pyrimidinyl, particularly phenyl;
L is —O— or -(1-3C)alkylO—;
R¹ is (1-6C)alkyl, optionally substituted by a substituent selected from hydroxy and (1-4C)alkoxy;
R² is selected from methylsulfonyl and azetidinylcarbonyl;
R³ is selected from fluoro, chloro, cyano, methoxy and carboxy;
R⁹, where present, is halo, methyl or methoxy;
R¹⁰ is absent;
R¹¹ is hydrogen;
m is 0 or 1;
n is 0 or 1.

8. A compound as claimed in claim 1, or a salt thereof, which is any one or more of the following:
2-{3-(benzyloxy)-5-[(1S)-2-methoxy-1-methylethoxy]phenyl}-1H-pyrrolo[2,3-b]pyridine;
2-{3-[(1S)-2-methoxy-1-methylethoxy]-5-[4-(methylsulfonyl)phenoxy]phenyl}-1H-pyrrolo[2,3-b]pyridine;
2-{3-{[2-(azetidin-1-ylcarbonyl)pyrimidin-5-yl]oxy}-5-[(1S)-2-methoxy-1-methylethoxy]phenyl}-1H-pyrrolo[2,3-b]pyridine and/or
8-[3-[(2S)-1-methoxypropan-2-yl]oxy-5-phenylmethoxy-phenyl]-2,5,7-triazabicyclo[4.3.0]nona-2,4,8,10-tetraene.

9. A pharmaceutical composition comprising a compound according to any one of the preceding claims, or a pharmaceutically-acceptable salt thereof, together with a pharmaceutically acceptable diluent or carrier.

10. A compound according to any one of the preceding claims, or a pharmaceutically-acceptable salt thereof for use as a medicament.

11. A compound according to any one of the preceding claims or a pharmaceutically-acceptable salt thereof for use in the preparation of a medicament for treatment of a disease mediated through GLK.

12. The use of a compound according to any one of the preceding claims, or a pharmaceutically-acceptable salt thereof in the preparation of a medicament for treatment of type 2 diabetes.

13. A method of treating GLK mediated diseases by administering an effective amount of a compound of Formula (I) as claimed in any one of the preceding claims or a pharmaceutically-acceptable salt thereof, to a mammal in need of such treatment.

14. The method of claim 13 wherein the GLK mediated disease is type 2 diabetes.

15. A process for the preparation of a compound of Formula (I), which comprises a process a) to h) (wherein the variables are as defined in claim 1 unless otherwise defined):
(a) reaction of a compound of Formula (III) with a compound of Formula (IV), 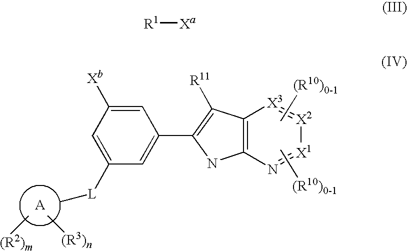
wherein X^{a} is a leaving group or an organometallic reagent and X^{b} is a hydroxyl group, or X^{a} is a hydroxyl group and X^{b} is a leaving group or an organometallic reagent, and wherein R¹ is as defined for a compound of formula (I), or is a protected version thereof; or
(b) reaction of a compound of Formula (V) with a compound of Formula (VI) 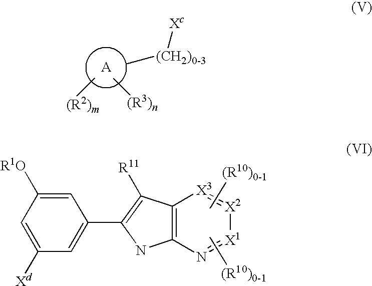
wherein X^{c} is a leaving group or an organometallic reagent and X^{d} is a hydroxyl group, or X^{c} is a hydroxyl group and X^{d} is a leaving group or an organometallic reagent, and wherein R¹ is as defined for a compound of formula (I), or is a protected version thereof; or
(c) reaction of a compound of Formula (VII) with a compound of Formula (VIII), 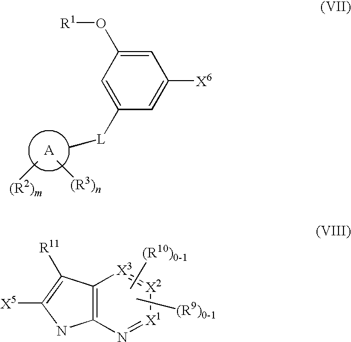
wherein X⁵ is a leaving group and X⁶ is an metal, or X⁶ is a leaving group and X⁵ is an metal; and wherein R¹ is as defined for a compound of formula (I) or is a protected version thereof; or
(d) reaction of a compound of formula (IX) with a compound of formula (X) and cyclisation in a one or two step reaction; 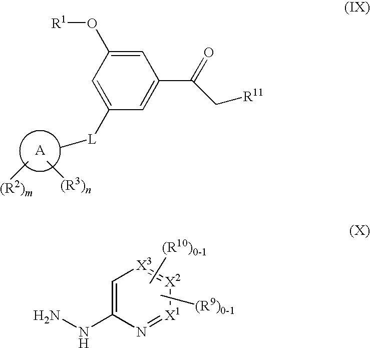
wherein R¹ and R¹¹ are as defined for a compound of formula (I) or a protected version thereof; or
e) reaction of a compound of formula (XI) with a compound of formula (XII) followed by cyclisation, 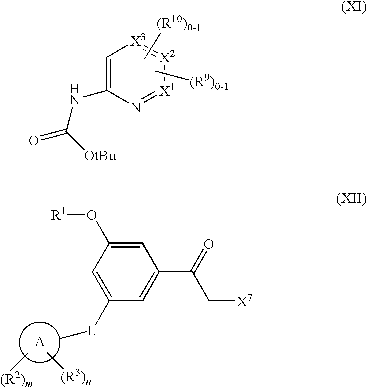
wherein X⁷ is a halogen, or other leaving group, such as —OR (wherein —OR represents an ester or activated ester), and wherein R¹ is as defined for a compound of formula (I) or is a protected version thereof; or
f) reaction of a compound of formula (XIII) with a compound of formula (XIV) followed by cyclisation, 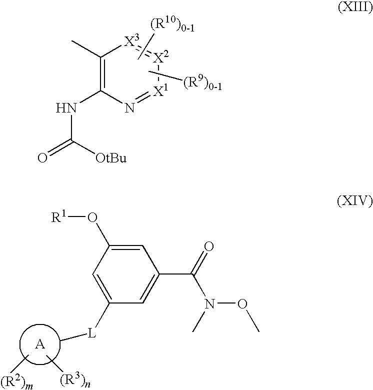
wherein R¹ is as defined for a compound of formula (I) or is a protected version thereof; or
g) reaction of a compound of formula (XV) with a compound of formula (XVI) in the presence of strong base; 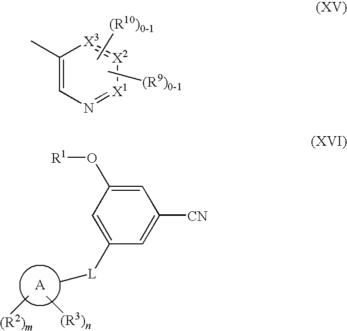
or
h) reaction of a compound of formula (XVII) with a compound of formula (XVIII) in the presence of strong base: 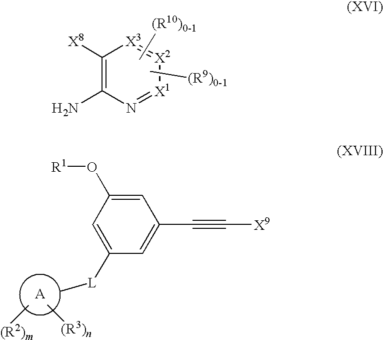
wherein X⁸ is halogen or other suitable leaving group and X⁹ is trimethyl silyl or R¹¹ (where R¹¹ is as defined for a compound of formula (I)), and R¹ is as defined for a compound of formula (I) or is a protected version thereof;
and thereafter, if necessary:
i) converting a compound of Formula (I) into another compound of Formula (I);
ii) removing any protecting groups; and/or
iii) forming a salt thereof.
